(19) United States
(12) Patent Application Publication
(10) Pub. No.: US 2006/0229335 A1
(43) Pub. Date: Oct. 12, 2006

(54) Diarylamine and arylheteroarylamine pyrazole derivatives as modulators of 5ht2a

(76) Inventors: Bradley Teegarden, San Diego, CA (US); Sonja Strah-Pleynet, San Diego, CA (US); Honnappa Jayakumar, San Diego, CA (US)

Correspondence Address: ARENA PHARMACEUTICALS, INC., 6166 NANCY RIDGE DRIVE, SAN DIEGO, CA, 92121, US

(21) Appl. No.: 10/540,650

(22) PCT Filed: Dec. 22, 2003
(86) PCT No.: PCT/US03/40844

§ 371 Date: Apr. 21, 2006

Related U.S. Application Data
(60) Provisional application No. 60/436,326, filed on Dec. 24, 2002.
(60) Provisional application No. 60/464,992, filed on Apr. 23, 2003.

(51) Int. Cl. A61K 31/4709; A61K 31/428; A61K 31/423; A61K 31/4178; A61K 31/415; C07D 417/02; C07D 413/02; C07D 403/02; C07D 231/12

(52) U.S. Cl. 514/314; 514/367; 514/397; 514/406; 546/159; 548/161; 548/217; 548/311.1; 548/377.1

(57) ABSTRACT

One aspect of the present invention relates to certain diarylamine and arylheteroarylamine pyrazole derivatives of Formula (A) and pharmaceutical compositions that modulate the activity of the human 5HT2A serotonin receptor. Compounds and pharmaceutical compositions are directed to methods useful in the prophylaxis or treatment of reducing platelet aggreagation, sleep disorders, coronary artery disease, myocardial infarction, transient ischemic attack, angina, stroke, atrial fibrillation, reducing the risk of blood clot formation, asthma or symptoms thereof, agitation or a symptom, behavioral disorders, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia and related disorders. Another aspect of the present invention is directed to the method of prophylaxis or treatment of 5HT2A serotonin receptor mediated disorders in combination with a dopamine D2 receptor antagonist such as haloperidol, administered separately or together.

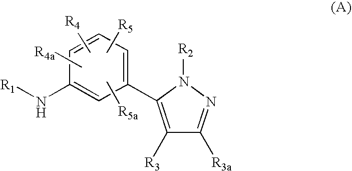

### FIELD OF THE INVENTION

One aspect of the present invention relates to certain diarylamine and arylheteroaryl-amine pyrazole derivatives as described herein and pharmaceutical compositions that modulate the activity of the human 5HT_{2A} serotonin receptor.

Compounds and pharmaceutical compositions are directed to methods useful in the prophylaxis or treatment of reducing platelet aggreagation, sleep disorders, coronary artery disease, myocardial infarction, transient ischemic attack, angina, stroke, atrial fibrillation, reducing the risk of blood clot formation, asthma or symptoms thereof, agitation or a symptom, behavioral disorders, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia and related disorders. Compounds and pharmaceutical compositions are also directed to methods useful in cardioprotection, for example to protect against heart failure and the like; neuroprotection, for example to protect against strokes and the like; and diabetic neuropathy.

Another aspect of the present invention is directed to the method of prophylaxis or treatment of 5HT_{2A} serotonin receptor mediated disorders in combination with a dopamine D2 receptor antagonist such as haloperidol, administered separately or together.

### BACKGROUND OF THE INVENTION

Serotonin Receptors

Receptors for serotonin (5-hydroxytryptamine, 5-HT) are an important class of G protein-coupled receptors. Serotonin is thought to play a role in processes related to learning and memory, sleep, thermoregulation, mood, motor activity, pain, sexual and aggressive behaviors, appetite, neurodegenerative regulation, and biological rhythms. Not surprisingly, serotonin is linked to pathophysiological conditions such as anxiety, depression, obsessive-compulsive disorders, schizophrenia, suicide, autism, migraine, emesis, alcoholism, and neurodegenerative disorders. With respect to on anti-psychotic treatment approaches focused on the serotonin receptors, these types of therapeutics can generally be divided into two classes, the “typical” and the “atypical.” Both have anti-psychotic effects, but the typicals also include concomitant motor-related side effects (extra pyramidal syndromes, e.g., lip-smacking, tongue darting, locomotor movement, etc). Such side effects are thought to be associated with the compounds interacting with other receptors, such as the human dopamine D2 receptor in the nigro-striatal pathway. Therefore, an atypical treatment is preferred. Haloperidol is considered a typical anti-psychotic, and clozapine is considered an atypical anti-psychotic.

Serotonin receptors are divided into seven subfamilies, referred to as 5-HT1 through 5-HT7, inclusive. These subfamilies are further divided into subtypes. For example, the 5-HT2 subfamily is divided into three receptor subtypes: 5-HT2A, 5-HT2B, and 5-HT2C. The human 5-HT2C receptor was first isolated and cloned in 1987, and the human 5-HT2A receptor was first isolated and cloned in 1990. These two receptors are thought to be the site of action of hallucinogenic drugs. Additionally, antagonists to the 5-HT2A and 5-RT2C receptors are believed to be useful in treating depression, anxiety, psychosis, and eating disorders.

U.S. Pat. No. 4,985,352 describes the isolation, characterization, and expression of a functional cDNA clone encoding the entire human 5-HT1C receptor (now known as the 5-HT2C receptor). U.S. Pat. No. 5,661,012 describes the isolation, characterization, and expression of a functional cDNA clone encoding the entire human 5-HT2A receptor.

Mutations of the endogenous forms of the rat 5-HT2A and rat 5-HT2C receptors have been reported to lead to constitutive activation of these receptors (5-HT2A: Casey, C. et al. (1996) Society for Neuroscience Abstracts, 22:699.10, hereinafter “Casey”; 5-HT2C: Herrick-Davis, K., and Teitler, M. (1996) Society for Neuroscience Abstracts, 22:699.18, hereinafter “Herrick-Davis 1”; and Herrick-Davis, K. et al. (1997) J. Neurochemistry 69(3): 1138, hereinafter “Herrick-Davis-2”). Casey describes a mutation of the cysteine residue at position 322 of the rat 5-HT2A receptor to lysine (C322K), glutamine (C322Q), and arginine (C322R) which reportedly led to constitutive activation. Herrick-Davis 1 and Herrick-Davis 2 describe mutations of the serine residue at position 312 of the rat 5-HT2C receptor to phenylalanine (S312F) and lysine (S312K), which reportedly led to constitutive activation.

### SUMMARY OF THE INVENTION

One aspect of the present invention pertains to certain diarylamine and arylheteroaryl-amine derivatives as shown in Formula (A):

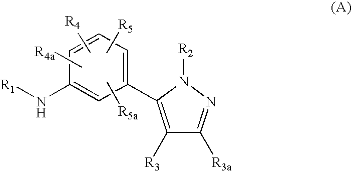

wherein:

i) R₁ is aryl or heteroaryl optionally substituted with 1 to 5 substituents selected independently from the group consisting of C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, amino, C₁₋₄ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol, nitro, phenoxy and phenyl; and wherein C₂₋₆ alkenyl, C₁₋₆ alkyl and C₂₋₄ alkynyl substituents may be optionally substituted with 1 to 5 substituents selected independently from the group consisting of C₁₋₅ acyl, C₁₋₅ acyloxy, C₂₋₆ alkenyl, C₁₋₄ alkoxy, C₁₋₄ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, amino, C₁₋₄ alkylamino, C₂₋₈ dialkylamino, carbo-C₁₋₅-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro; or two adjacent substituents together with the ring carbons to which they are bonded form a C₅₋₇ cycloalkyl optionally replaced with 1 to 2 oxygen atoms;

ii) R₂ is C₁₋₆ alkyl, C₂₋₆ alkenyl, C₂₋₆ alkynyl or C₃₋₇ cycloalkyl;

iii) R₃ is H, C₂₋₆ alkenyl, C₁₋₆ alkyl, C₁₋₅ alkylcarboxamide, C₂₋₆ alkynyl, C₁₋₄ alkylsulfonamide, carbo-C₁₋₆-alkoxy, carboxamide, carboxy, cyano, C₃₋₇ cycloalkyl, C₂₋₈ dialkylcarboxamide, halogen, heteroaryl or phenyl; and wherein C₂₋₆ alkenyl, C₁₋₆ alkyl, C₂₋₆ alkynyl, C₁₋₄ alkylsulfonamide, C₃₋₇ cycloalkyl, heteroaryl or phenyl may be optionally substituted with 1 to 5 substituents selected independently from the group consisting of C₂₋₆ alkenyl, C₁₋₆ alkyl, C₁₋₄ alkoxy, amino, C₁₋₄ alkylamino, C₂₋₆ alkynyl, C₂₋₈ dialkylamino, halogen, C₁₋₄ haloalkoxy, C₁₋₄ haloalkyl, hydroxyl and thiol;

iv) R₃ₐ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₄ haloalkylsulfinyl, C₁₋₆ haloalkylsulfonyl, C₁₋₆ haloalkylthio, hydroxyl, thiol, nitro and sulfonamide; and

v) R₄, R₄ₐ, R₅ and R₅ₐ are independently H, C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfonyl, C₁₋₄ haloalkylsulfonyl, C₁₋₄ haloalkylthio, hydroxyl, thiol, 5 or 6 membered-heteroaryl, nitro, phenyl or NR₆R₇, and where the 5 or 6 membered-heteroaryl or phenyl is optionally substituted with a substituents selected from the group consisting of H, C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfonyl, C₁₋₄ alkylsulfonyl, C₁₋₄ alkylthio, C₁₋₄ alkylureyl, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro;
wherein:
   R₆ and R₇ are independently selected from the group consisting of H, C₁₋₆ alkyl, C₂₋₆ alkenyl, C₃₋₇ cycloalkyl, phenyl and benzyl group; wherein each said C₁₋₆ alkyl, C₂₋₆ alkenyl, C₃₋₇ cycloalkyl, phenyl and benzyl group is optionally substituted with 1 to 5 substituents selected independently from the group consisting of H, C₁₋₅ acyl, C₂₋₆ alkenyl, C₁₋₄ alkoxy, C₁₋₆ alkyl, C₁₋₅ alkylcarboxamide, C₁₋₄ alkylthio, carbo-C₁₋₆-alkoxy, amino, C₁₋₄ alkylamino, C₂₋₈ dialkylamino, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro; or
   R₆ and R₇ together with the nitrogen to which they are bonded form a 5, 6 or 7 membered cyclic structure which can be saturated or unsaturated and can contain up to four heteroatoms selected from O, NR₈ or S and said cyclic structure may be optionally substituted with 1 to 5 substituents selected independently from the group consisting of H, C₁₋₅ acyl, C₂₋₆ alkenyl, C₁₋₄ alkoxy, C₁₋₆ alkyl, C₁₋₅ alkylcarboxamide, C₁₋₄ alkylthio, carbo-C₁₋₆-alkoxy, amino, C₁₋₄ alkylamino, C₂₋₈ dialkylamino, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfonyl, C₁₋₄ haloalkylsulfonyl, C₁₋₄ haloalkylthio, hydroxyl, thiol and nitro;
   R₈ is H or C₁₋₆ alkyl; or

a pharmaceutically acceptable salt, hydrate or solvate thereof.

One aspect of the present invention encompasses certain diarylamine and arylheteroaryl-amine derivatives of Formula (A) wherein R₃ₐ, R₄ₐ, and R₅ₐ are each H and is represented by Formula (I): 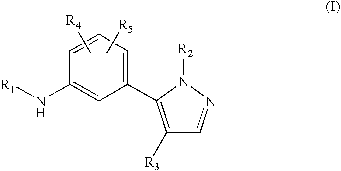
or a pharmaceutically acceptable salt, hydrate or solvate thereof, wherein R₁, R₂, R₃, R₄ and R₅ are as defined herein.

Some embodiments of the present invention include pharmaceutical compositions comprising compounds as described herein and a pharmaceutically acceptable carrier.

Some embodiments of the present invention are methods for modulating the activity of a human 5HT_{2A} serotonin receptor comprising contacting the receptor with a compound or a pharmaceutical composition as described herein.

Some embodiments of the present invention are methods for the prophylaxis or treatment of reducing platelet aggregation in an individual comprising administering to the individual in need of such prophylaxis or treatment a compound or a pharmaceutical composition as described herein

Some embodiments of the present invention are methods for the prophylaxis or treatment of an indication selected from the group consisting of coronary artery disease, myocardial infarction, transient ischemic attack, angina, stroke, and atrial fibrillation in an individual comprising administering to the individual in need of such treatment or prophylaxis a compound or a pharmaceutical composition as described herein.

Some embodiments of the present invention are methods for the prophylaxis or treatment of reducing a risk of blood clot formation in an angioplasty or coronary bypass surgery individual, comprising administering to the individual in need of such prophylaxis or treatment a compound or a pharmaceutical composition as described herein.

Some embodiments of the present invention are methods for the prophylaxis or treatment of reducing risk of blood clot formation in an individual suffering from atrial fibrillation, comprising administering to the individual in need of such prophylaxis or treatment a compound or a pharmaceutical composition as described herein.

Some embodiments of the present invention are methods for the prophylaxis or treatment of asthma in an individual, comprising administering to the individual in need of such prophylaxis or treatment a compound or a pharmaceutical composition as described herein.

Some embodiments of the present invention are methods for the prophylaxis or treatment of a symptom of asthma in an individual, comprising administering to the individual in need of such prophylaxis or treatment a compound or a pharmaceutical composition as described herein.

Some embodiments of the present invention are methods for the prophylaxis or treatment of agitation or a symptom thereof in an individual, comprising administering to the individual in need of such prophylaxis or treatment a compound or a pharmaceutical composition as described herein. In some embodiments the individual is a cognitively intact elderly individual.

Some embodiments of the present invention are methods for prophylaxis or treatment of agitation or a symptom thereof in an individual suffering from dementia, comprising administering to the individual in need of such prophylaxis or treatment a compound or a pharmaceutical composition as described herein. In some embodiments the dementia is due to a degenerative disease of the nervous system. In some embodiments the dementia is Alzheimers disease, Lewy Body, Parkinson's disease, or Huntington's disease. In some embodiments the dementia is due to diseases that affect blood vessels. In some embodiments the dementia is due to stroke or multi-infarct dementia.

Some embodiments of the present invention are methods for the prophylaxis or treatment of an individual suffering from at least one of the indications selected from the group consisting of behavioral disorder, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia comprising administering to the individual in need of such prophylaxis or treatment a dopamine D2 receptor antagonist and a compound or a pharmaceutical composition as described herein. In some embodiments the dopamine D2 receptor antagonist is haloperidol.

Some embodiments of the present invention are methods for the prophylaxis or treatment of an individual with infantile autism, Huntington's chorea, or nausea and/or vomiting from chemotherapy or chemotherapeutic antibodies comprising administering to the individual in need of such prophylaxis or treatment a dopamine D2 receptor antagonist and a compound or a pharmaceutical composition as described herein. In some embodiments the dopamine D2 receptor antagonist is haloperidol.

Some embodiments of the present invention are methods for the prophylaxis or treatment of schizophrenia in an individual, comprising administering to the individual in need of such prophylaxis or treatment a dopamine D2 receptor antagonist and a compound or a pharmaceutical composition as described herein. In some embodiments the dopamine D2 receptor antagonist is haloperidol.

Some embodiments of the present invention are methods for the prophylaxis or treatment of alleviating negative symptoms of schizophrenia induced by the administration of haloperidol to an individual suffering from schizophrenia, comprising administering to the individual in need of such prophylaxis or treatment a compound or a pharmaceutical composition as described herein.

Some embodiments of the present invention include methods for the prophylaxis or treatment wherein haloperidol and the compound or pharmaceutical composition are administered in separate dosage forms.

Some embodiments of the present invention include methods for the prophylaxis or treatment wherein haloperidol and said compound or pharmaceutical composition are administered in a single dosage form.

Some embodiments of the present invention include methods for the prophylaxis or treatment of a sleep disorder in an individual comprising administering to said individual in need of such prophylaxis or treatment a compound or a pharmaceutical composition as described herein.

Some embodiments of the present invention are compounds described herein for use in a method of treatment of the human or animal body by therapy.

Some embodiments of the present invention are compounds described herein for use in a method for the prophylaxis or treatment of reducing platelet aggregation in the human or animal body by therapy.

Some embodiments of the present invention are compounds described herein for use in a method for the prophylaxis or treatment of an indication selected from the group consisting of coronary artery disease, myocardial infarction, transient ischemic attack, angina, stroke, and atrial fibrillation in the human or animal body by therapy.

Some embodiments of the present invention are compounds described herein for use in a method for the prophylaxis or treatment of reducing risk-of blood clot formation in an angioplasty or coronary bypass surgery in the human or animal body by therapy.

Some embodiments of the present invention are compounds described herein for use in a method for the prophylaxis or treatment of reducing risk of blood clot formation in the human or animal body by therapy.

Some embodiments of the present invention are compounds described herein for use in a method for the prophylaxis or treatment of asthma in the human or animal body by therapy.

Some embodiments of the present invention are compounds described herein for use in a method for the prophylaxis or treatment of a symptom of asthma in the human or animal body by therapy.

Some embodiments of the present invention are compounds described herein for use in a method for the prophylaxis or treatment of agitation or a symptom thereof in the human or animal body by therapy.

Some embodiments of the present invention are compounds described herein for use in a method for the prophylaxis or treatment of at least one of the indications selected from the group consisting of behavioral disorder, drug induced psychosis, excitative psychosis, Gilles de la Tourette's syndrome, manic disorder, organic or NOS psychosis, psychotic disorder, psychosis, acute schizophrenia, chronic schizophrenia and NOS schizophreniain in the human or animal body by therapy.

Some embodiments of the present invention are compounds described herein for use in a method for the prophylaxis or treatment of a sleep disorder in the human or animal body by therapy.

One aspect of the present invention pertains to the use of a compound, as described herein, for the manufacture of a medicament for use in the treatment of a 5HT_{2A} mediated disorder.

One embodiment of the present invention pertains to the use of a compound for the production of a medicament for use in the prophylaxis or treatment of a 5HT_{2A} mediated disorder wherein the disorder is platelet aggregation.

One embodiment of the present invention pertains to the use of a compound for the production of a medicament for use in the prophylaxis or treatment of a 5HT_{2A} mediated disorder wherein the disorder is selected from the group consisting of coronary artery disease, myocardial infarction, transient ischemic attack, angina, stroke, and atrial fibrillation.

One embodiment of the present invention pertains to the use of a compound for the production of a medicament for use in the prophylaxis or treatment of a 5HT_{2A} mediated disorder wherein the disorder is a blood clot formation in an angioplasty or coronary bypass surgery individual.

One embodiment of the present invention pertains to the use of a compound for the production of a medicament for use in the prophylaxis or treatment of a 5HT_{2A} mediated disorder wherein the disorder is a blood clot formation in an individual suffering from atrial fibrillation.

One embodiment of the present invention pertains to the use of a compound for the production of a medicament for use in the prophylaxis or treatment of a 5HT_{2A} mediated disorder wherein the disorder is asthma.

One embodiment of the present invention pertains to the use of a compound for the production of a medicament for use in the prophylaxis or treatment of a 5HT_{2A} mediated disorder wherein the disorder is a symptom of asthma.

One embodiment of the present invention pertains to the use of a compound for the production of a medicament for use in the prophylaxis or treatment of a 5HT_{2A} mediated disorder wherein the disorder is agitation or a symptom thereof in an individual. In some embodiments the individual is a cognitively intact elderly individual.

One embodiment of the present invention pertains to the use of a compound for the production of a medicament for use in the prophylaxis or treatment of a 5HT_{2A} mediated disorder wherein the disorder is agitation or a symptom thereof in an individual suffering from dementia. In some embodiments the dementia is due to a degenerative disease of the nervous system. In some embodiment the dementia is Alzheimers disease, Lewy Body, Parkinson's disease, or Huntington's disease. In some embodiments the dementia is due to diseases that affect blood vessels. In some embodiments the dementia is due to stroke or multi-infract dementia.

One embodiment of the present invention pertains to the use of a compound for the production of a medicament for use in the prophylaxis or treatment of a 5HT_{2A} mediated disorder further comprising a dopamine D2 receptor antagonist wherein the disorder is selected from the group consisting of a behavioral disorder, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia. In some embodiments the dopamine D2 receptor antagonist is haloperidol.

One embodiment of the present invention pertains to the use of a compound for the production of a medicament for use in the prophylaxis or treatment of a 5RT_{2A} mediated disorder further comprising a dopamine D2 receptor antagonist wherein the disorder is infantile autism, Huntington's chorea, or nausea and vomiting from chemotherapy or chemotherapeutic antibodies. In some embodiments the dopamine D2 receptor antagonist is haloperidol.

One embodiment of the present invention pertains to the use of a compound for the production of a medicament for use in the prophylaxis or treatment of a 5HT_{2A} mediated disorder further comprising a dopamine D2 receptor antagonist wherein the disorder is schizophrenia. In some embodiments the dopamine D2 receptor antagonist is haloperidol.

One embodiment of the present invention pertains to the use of a compound for the production of a medicament for use in the prophylaxis or treatment of a 5HT_{2A} mediated disorder wherein the disorder is a negative symptom or symptoms of schizophrenia induced by the administration of haloperidol.

One embodiment of the present invention pertains to the use of a compound for the production of a medicament for use in the prophylaxis or treatment of a 5HT_{2A} mediated disorder wherein the haloperidol and the compound or pharmaceutical composition are administered in separate dosage forms.

One embodiment of the present invention pertains to the use of a compound for the production of a medicament for use in the prophylaxis or treatment of a 5HT_{2A} mediated disorder wherein the haloperidol and the compound or pharmaceutical composition are administered in a single dosage form.

One embodiment of the present invention pertains to the use of a compound for the production of a medicament for use in the prophylaxis or treatment of a sleep disorder.

One aspect of the present invention is a process for preparing a composition comprising admixing a compound, as described herein, and a pharmaceutically acceptable carrier.

These and other aspects of the invention disclosed herein will be set forth in greater detail as the patent disclosure proceeds.

### DETAILED DESCRIPTION

The present invention provides compounds that are useful, for example, for the prophylaxis or treatment of 5HT_{2A} related disorders. The present invention may be understood more readily by reference to the following detailed description of the invention and the Examples included therein and to the Figures and their previous and following description. It is also to be understood that the terminology used herein is for the purpose of describing particular embodiments only and is not intended to be limiting.

### DEFINITIONS

In the specification and Formulae described herein the following terms are hereby defined.

AGONISTS shall mean moieties that activate the intracellular response when they bind to the receptor, or enhance GTP binding to membranes.

AMINO ACID ABBREVIATIONS used herein are set out in TABLE 1:

TABLE 1

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

PARTIAL AGONISTS shall mean moieties which activate the intracellular response when they bind to the receptor to a lesser degree/extent than do agonists, or enhance GTP binding to membranes to a lesser degree/extent than do agonists.

ANTAGONIST shall mean moieties that competitively bind to the receptor at the same site as the agonists but which-do not activate the intracellular response initiated by the active form of the receptor, and can thereby inhibit the intracellular responses by agonists or partial agonists. ANTAGONISTS do not diminish the baseline intracellular response in the absence of an agonist or partial agonist.

Chemical Group, Moiety or Radical:

A chemical group, moiety or radical of a compound of the present invention, as used in the specification and concluding claims, refers to a structural fragment of the compound.

The term “C₁₋₅ acyl” denotes an allyl radical bonded directly to a carbonyl group [i.e., —C(O)—] wherein the definition of alkyl has the same definition as described herein; some examples include formyl, acetyl, propionyl, butanoyl, iso-butanoyl, pentanoyl, and the like.

The term “C₁₋₅ acyloxy” denotes an acyl radical attached to an oxygen atom wherein acyl has the same definition has described herein; some examples include acetyloxy, propionyloxy, butanoyloxy, iso-butanoyloxy and the like.

The term “C₂₋₄ alkenyl” denotes a radical containing 2 to 6 carbons wherein at least one carbon-carbon double bond is present, some embodiments are 2 to 4 carbons, some embodiments are 2 to 3 carbons, and some embodiments have 2 carbons. Both E and Z isomers are embraced by the term “alkenyl.” Furthermore, the term “alkenyl” includes di- and tri-alkenyls. Accordingly, if more than one double bond is present then the bonds may be all E or Z or a mixtures of E and Z. Examples of an alkenyl include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl and the like.

The term “C₁₋₄ alkoxy” as used herein denotes a radical alkyl, as defined herein, attached directly to an oxygen atom. In some embodiments, the alkoxy group contains 1 to 3 carbons (i.e., C₁₋₃ alkoxy). Examples of an alkoxy group include methoxy, ethoxy, n:-propoxy, iso-propoxy, n-butoxy, t-butoxy, iso-butoxy and the like.

The term “C₁₋₆ alkyl” denotes a straight or branched carbon radical containing 1 to 6 carbons, some embodiments are 1 to 4 carbons, some embodiments are 1 to 3 carbons, and some embodiments are 1 or 2 carbons. Examples of an alkyl include methyl, ethyl, n-propyl, iso-propyl, n-butyl, sec-butyl, t-butyl, amyl, t-amyl, n-pentyl, n-hexyl and the like.

The term “C₁₋₅ alkylcarboxamido” denotes a single alkyl group attached to either the nitrogen or carbonyl of an amide group, wherein alkyl has the same definition as found herein. The C₁₋₅ alkylcarboxamido may be represented by the following formulae: 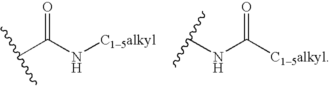
Examples include N-methylcarboxamide, N-ethylcarboxamide, N-(iso-propyl)carboxamide and the like.

The term “C₁₋₄ alkylsulfinyl” denotes an alkyl radical attached to a sulfoxide radical of the formula: —S(O)— wherein the alkyl radical has the same definition as described herein. Examples include methylsulfinyl, ethylsulfinyl and the like.

The term “C₁₋₄ alkylsulfonamide” denotes an alkyl radical attached to the nitrogen or sulfur of a sulfonamide group of the formula: —S(O)₂NH— wherein the alkyl radical has the same definition as described herein and a C₁₋₄ alkylsulfon-amide may be represented by the following formulae:

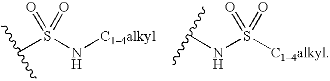

The term “C₁₋₄ alkylsulfonyl” denotes an alkyl radical attached to a sulfone radical of the formula: —S(O)₂— wherein the alkyl radical has the same definition as described herein. Examples include methylsulfonyl, ethylsulfonyl and the like.

The term “C₁₋₄ alkylthio” denotes an alkyl radical attached to a sulfide group of the formula: —S— wherein the alkyl radical has the same definition as described herein. Examples include methylsulfide (i.e., CH₃S—), ethylsulfide, isopropylsulfide and the like.

The term “C₁₋₆ alkylureyl” denotes the group of the formula: —NC(O)N— wherein one are both of the nitrogens are substituted with the same or different alkyl group wherein alkyl has the same definition as described herein. Examples of an alkylureyl include, CH₃NHC(O)NH—, NH₂C(O)NCH₃—, (CH₃)₂N(O)NH—, (CH₃)₂N(O)NH—, (CH₃)₂N(O)NCH₃—, CH₃CH₂NHC(O)NH—, CH₃CH₂NHC(O)NCH₃—, and the like.

The term “C₂₋₆ alkynyl” denotes a radical containing 2 to 6 carbons and at least one carbon-carbon triple bond, some embodiments are 2 to 4 carbons, some embodiments are 2 to 3 carbons, and some embodiments have 2 carbons. Examples of an alkynyl include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “amino” denotes the group —NH₂.

The term “C₁₄ alkylamino” denotes one alkyl radical attached to an amino radical wherein the alkyl radical has the same meaning as described herein. Some examples include methylamino, ethylamino, propylamino and the like.

The term “aryl” denotes an aromatic ring radical containing 6 to 10 ring carbons. Some examples include phenyl, naphthyl and the like.

The term “arylalkyl” defines a C₁-C₄ alkylene, such as —CH₂—, —CH₂CH₂— and the like, which is further substituted with an aryl group. Examples of an “arylalkyl” include benzyl, phenethylene and the like.

The term “arylcarboxamido” denotes a single aryl group attached to the amine of an amide, wherein aryl has the same definition as found herein. The example is N-phenylcarboxamide.

The term “arylureyl” denotes the group —NC(O)N— where one of the nitrogens are substituted with an aryl.

The term “benzyl” denotes the group CH₂C₆H₅.
The term “carbo-C₁₋₆-alkoxy” refers to an alkyl ester of a carboxylic acid, wherein the alkyl group is C₁₋₆. Examples include carbomethoxy, carboethoxy, carboisopropoxy and the like.

The term “carboxamide” denotes the group —CONH₂.

The term “carboxy” or “carboxyl” denotes the group —CO₂H; also referred to as a carboxylic acid.

The term “cyano” denotes the group —CN.
The term —C₃₋₇ cycloalkyl” denotes a saturated ring radical containing 3 to 7 carbons; some embodiments contain 3 to 6 carbons; some embodiments contain 3 to 5 carbons, some embodiments contain 3 to 4 carbons. Examples include cyclopropyl, cyclobutyl, cyclopentyl, cyclopenyl, cyclohexyl, cycloheptyl and the like.

The term “C₂₋₆ dialkylamino” denotes an amino substituted with two of the same or different alkyl radicals wherein alkyl radical has the same definition as described herein. Some examples include dimethylamino, methylethylamino, diethylamino and the like.

The term “C₂₋₈ dialkylcarboxamide” denotes two alkyl radicals, that are the same or different, attached to an amide group, wherein alkyl has the same definition as described herein. A C₂₋₈ dialkylcarboxamide may be represented by the following groups: 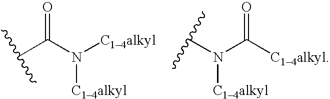
Examples of a dialkylcarboxamide include N,N-dimethylcarboxamide, N-methyl-N-ethylcarboxamide and the like.

The term “C₂₋₄ dialkylsulfonamide” denotes two alkyl radicals attached independently to the nitrogen or sulfur of a sulfonamide group of the formula: S(O)₂N wherein the alkyl radicals have the same definition as described herein and may be the same or different; a C₂₋₈ alkylsulfonamide may be represented by the following formulae:

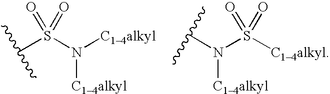

The term “halo” or “halogen” denotes to a fluoro, chloro, bromo or iodo atom.

The term “C₁₋₄ haloalkoxy” denotes a haloalkyl, as defined herein, that is directly attached to an oxygen to form a difluoromethoxy, trifluoromethoxy, 2,2,2-trifluoroethoxy, pentafluoroethoxy and the like.

The term “C₁₋₄ haloalkyl” denotes an alkyl group, defined herein, wherein the alkyl is substituted with one halogen up to completely substituted with halogens and may be represented by the formula Cₙhalogen₂ₙ₊₁; when more than one halogen is present they may be the same or different and selected from F, Cl, Br or L In some embodiments, the haloalkyl contains 1 to 3 carbons (i.e., C₁₋₃ haloalkyl). Examples of an haloalkyl group include fluoromethyl, difluoromethyl, trifluoromethyl, chlorodifluoromethyl, bromodifluoromethyl, 2,2,2-trifluoroethyl, pentafluoroethyl and the like.

The term “C₁₋₄ haloalkylsulfinyl” denotes a haloalkyl radical attached to a sulfoxide of the formula: —S(O)— wherein the alkyl radical has the same definition as described herein. Examples include trifluoromethylsulfinyl, chlorodifluoro-methylsulfinyl, 2,2,2-trifluoroethylsulfinyl, 2,2-difluoroethylsulfinyl and the like.

The term “C₁₋₄ haloalkylsulfonyl” denotes a haloalkyl radical attached to a sulfone of the formula: —S(O)₂— wherein haloalkyl has the same definition as described herein. Examples include trifluoromethylsulfonyl, chlorodifluoro-methylsulfonyl, 2,2,2-trifluoroethylsulfonyl, 2,2-difluoroethylsulfonyl and the like.

The term “C₁₋₄ haloalkylthio” denotes an alkylthio radical substituted with one or more halogens. Examples include trifluoromethylthio, 1,1-difluoroethylthio, 2,2,2-trifluoroethylthio and the like.

The term “heteroaryl” denotes an aromatic ring system that may be a single ring, such as 5 or 6-membered ring containing carbons and at least one ring heteroatom selected from O, S and N or a heteroaryl group may be a 5 or 6-membered ring fused with a phenyl or another heteroaryl ring system. Examples of heteroaryl groups include, but not limited to, quinolinyl, benzoxazolyl, benzimidazolyl, benzothiazolyl, quinazolinyl, pyrimidinyl, pyridyl, pyrazinyl, pyridazinyl, triazinyl, isoquinolinyl, quinazolinyl, quinoxalinyl and the like.

The term “heterocyclic” denotes a 5, 6 or 7 membered non-aromatic carbon ring wherein at least one ring carbon is replaced by one, two or three heteroatoms, such as, piperidinyl, morpholinyl, piperzinyl, pyrrolidinyl, and the like.

The term “heterocycliccarboxamido” denotes a heterocyclic group with a ring nitrogen where the ring nitrogen is bonded directly to the carbonyl forming an amide. Examples include: 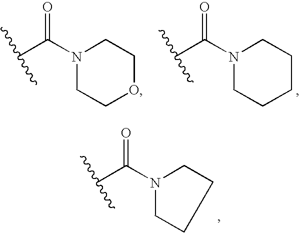
and the like

The term “hydroxyl” refers to the group —OH.

The term “nitro” refers to the group —NO₂.

The term “perfluoroalkyl” denotes the group of the formula —CₙF₂ₙ₊₁; stated differently, a perfluoroalkyl is an alkyl as defined herein herein wherein the alkyl is fully substituted with fluorine atoms and is therefore considered a subset of haloalkyl. Examples of perfluoroalkyls include CF₃, CF₂CF₃, CF₂CF₂CF₃, CF(CF₃)₂, CF₂CF₂CF₂CF₃, CF₂CF(CF₃)₂, CF(CF₃)CF₂CF₃ and the like.

The term “phenoxy” refers to the group C₆H₅O—.

The term “phenyl” refers to the group C₆H₅—.

The term “thiol” denotes the group —SH.

COMPOSITION shall mean a material comprising at least two compounds or two components; for example, and not limitation, a Pharmaceutical Composition is a Composition.

COMPOUND EFFICACY shall mean a measurement of the ability of a compound to inhibit or stimulate receptor functionality, as opposed to receptor binding affinity.

CONTACT or CONTACTING shall mean bringing at least two moieties together, whether in an in vitro system or an in vivo system.

INDIVIDUAL as used herein refers to any animal, including mammals, preferably mice, rats, other rodents, rabbits, dogs, cats, swine, cattle, sheep, horses, or primates, and most preferably humans.

INHIBIT or INHIBITING, in relationship to the term “response” shall mean that a response is decreased or prevented in the presence of a compound as opposed to in the absence of the compound.

IN NEED OF PROPHYLAXIS OR TREATMENT as used herein refers to a judgement made by a caregiver (e.g. physician, nurse, nurse practitioner, etc. in the case of humans; veterinarian in the case of animals, including non-human mammals) that an individual requires or will benefit from prophylaxis or treatment. This judgement is made based on a variety of factors that are in the realm of a caregiver's expertise, but that includes the knowledge that the individual or animal is ill, or will be ill, as the result of a condition that is treatable by the compounds of the invention.

INVERSE AGONISTS shall mean moieties that bind the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

LIGAND shall mean an endogenous, naturally occurring molecule specific for an endogenous, naturally occurring receptor.

As used herein, the terms MODULATE or MODULATING shall mean to refer to an increase or decrease in the amount, quality, response or effect of a particular activity, function or molecule. For example, Compounds which modulate/capable of modulating the 5HT_{2A} activity include agonists, inverse agonists, antagonists, inhibitors, activators, and compounds which directly or indirectly affect regulation of the 5HT_{2A} activity.

PHARMACEUTICAL COMPOSITION shall mean a composition comprising at least one active ingredient, whereby the composition is amenable to investigation for a specified, efficacious outcome in a manual (for example, and not limitation, a human). Those of ordinary skill in the art will understand and appreciate the techniques appropriate for determining whether an active ingredient has a desired efficacious outcome based upon the needs of the artisan.

STIMULATE or STIMULATING, in relationship to the term “response” shall mean that a response is increased in the presence of a compound as opposed to in the absence of the compound.

### COMPOUND OF THE PRESENT INVENTION

One aspect of the present invention pertains to certain diarylamine and arylheteroaryl-amine derivatives as shown in Formula (A): 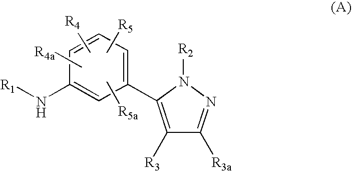
or a pharmaceutically acceptable salt, hydrate or solvate thereof, wherein R₁, R₂, R₃, R₃ₐ, R₄, R₄ₐ, R₅, and R₅ₐ are as described herein.

One aspect of the present invention encompasses certain diarylamine and arylheteroaryl-amine derivatives of Formula (A) wherein R₃ₐ, R₄ₐ, and R₅ₐ are each H and is represented by Formula (I): 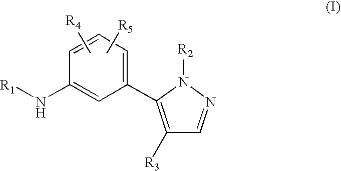
or a pharmaceutically acceptable salt, hydrate or solvate thereof, wherein R₁, R₂, R₃, R₄ and R₅ are as defined herein.

In some embodiments of the present invention, the compound is not a compound of Formula (Ia):

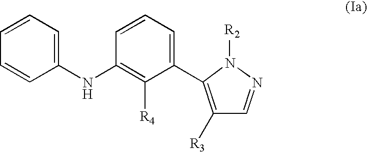

wherein:

R₂ is C₁₋₆ alkyl;

R₃ is: H, or halogen atom, an C₁₋₄ alkyl, C₂₋₄ alkenyl or C₂₋₄ alkynyl, optionally substituted with halogen, a phenyl optionally substituted with C₁₋₃ alkyl, C₁₋₃ haloalkyl or C₁₋₃ alkoxy, or a carboxy; and

R₄ is H, cyano, halogen atom, hydroxyl, thiol, nitro or NR₆R₇; wherein R₆ and R₇ are independently C₁₋₆ alkyl or phenyl.

Formula (Ia) explicitly shows the R₁ as a phenyl group that is substituted with only hydrogens atoms.

In some embodiments, compounds of the present invention are when R₁ is aryl and may be represented by Formulae (IIIa) and (IIIb): 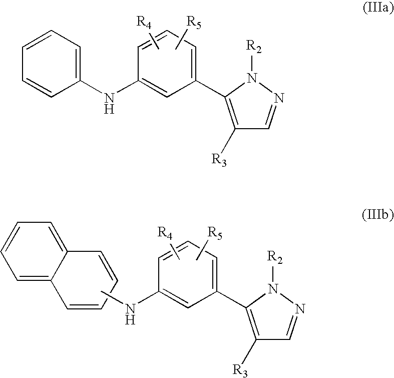
the R₁ phenyl group of Formula (IIIa) and R₁ naphthyl group of Formula (IIIb) may be optionally substituted with 1 to 5 substituents selected independently from the group consisting of C₁₋₅ acyl, C₁₋₄ alkoxy, C₁₋₆ alkyl, C₁₋₅ alkylcarboxamide, C₁₋₄ alkylsulfinyl, C₁₋₄ alkylsulfonyl, C₁₋₄ alkylthio, amino, C₁₋₄ alkylamino, C₂₋₈ dialkylamino, carboxamide, carboxy, carbo-C₁₋₆-alkoxy, cyano, C₂₋₈ dialkylcarboxamide, halogen, C₁₋₄ haloalkoxy, C₁₋₄ haloalkyl, hydroxyl, thiol, nitro and phenoxy. In some embodiments the C₁₋₆ alkyl substituents is optionally substituted with 1 to 3 substituents selected from the group consisting of C₁₋₄ alkoxy, C₁₋₅ alkylcarboxamide, amino, C₁₋₄ alkylamino, C₂₋₈ dialkylamino, halogen, C₁₋₄ haloalkoxy, C₁₋₄ haloalkyl, hydroxyl and thiol. It is understood that in some embodiments, R₁ is naphth-1-yl, Formula (IIIc), and naphth-2-yl, Formula (IIId), accordingly, both aryl groups are embraced in Formula (IIIb).

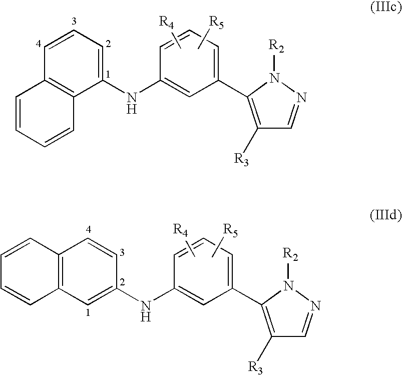

In some embodiments R₁ is aryl, such as phenyl [Formula (IIIa)] or naphthyl [Formula (IIIb)], and optionally substituted with 1 to 5 substituents selected independently from the group consisting of NO₂, F, Cl, Br, I, CF₃, CF₂CF₃, OCH₃, OCH₂CH₃, OCF₃, OCF₂CF₃, SCH₃, SCH₂CH₃, S(O)CH₃, S(O)CH₂CH₃, S(O)₂CH₃, S(O)₂CH₂CH₃, CO₂H, CN, COCH₃, COCH₂CH₃, CH₃, CH₂CH₃, NHCOCH₃, CH₂OH and OC₆H₅. In some embodiments R₁ is aryl, such as phenyl [Formula (IlIa)] or naphthyl [Formula (IIIb)], and optionally substituted with 1 to 3 substituents selected independently from the group consisting of NO₂, F, Cl, Br, L CF₃, OCH₃, OCF₃SCH₃, S(O)CH₃, S(O CH₃, CN, COCH₃, CH₃, CH₂OH and OC₆H₅. In some embodiments R₁ is aryl, such as phenyl [Formula (IIIa)] or naphthyl [Formula (IIIb)], and optionally substituted with 1 to 3 substituents selected independently from the group consisting of NO₂, F, Cl, Br, I, CF₃, OCH₃, OCF₃, SCH₃, S(O)CH₃, S(O)₂CH₃ CN and CH₃. In some embodiments of the invention R₁ is aryl, such as phenyl [Formula (IIIa)] or naphthyl [Formula (IIIb)], and optionally substituted with 1 to 3 substituents selected independently from the group consisting of F, Cl, Br, I, CF₃, OCH₃ and OCF₃.

In some embodiments, compounds of the present invention are when aryl is phenyl, as represented herein as [Formula (IIIa)], and optionally substituted with 1 to 3 substituents selected independently from the group consisting of F, Cl, Br, I, CF₃, OCH₃ and OCF₃.

In some embodiments, compounds of the present invention are when aryl is 2-naphthyl, as represented in [Formula (IIId)], and optionally substituted with 1 to 3 substituents selected independently from the group consisting of F, Cl, Br, I, CF₃, OCH₃ and OCF₃.

In some embodiments, compounds of the present invention are when R₁ is aryl and two adjacent substituents together with the ring carbons to which they are bonded form a C₅₋₇ cycloalkyl optionally replaced with 1 to 2 oxygen atoms. In some embodiments the C₅₋₇ cycloalkyl together with the aryl may be represented by Formulae (IIIe)-(IIIg): 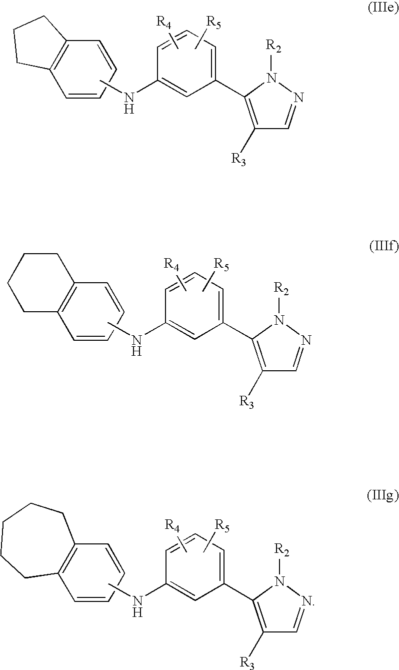
In some embodiments, the C₅₋₇ cycloalkyl is replaced with 1 to 2 oxygen atoms, accordingly, 1 or 2 cycloalkyl ring carbons is replaced with an oxygen atom. In some embodiments, 1 oxygen atom is present as shown in Formula (IIIh)-(IIIj): 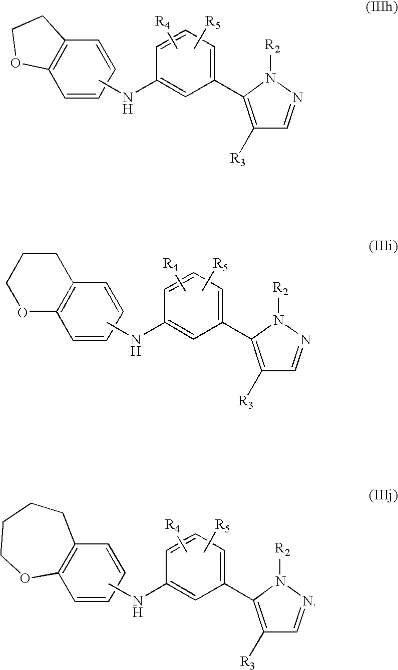
In some embodiments, 2 oxygen atoms are present in the ring. In some embodiments, R₁ is a 3,4-methylenedioxyphenyl or 3,4-ethylenedioxyphenyl group represented by Formula (IIIk) and (IIIm).

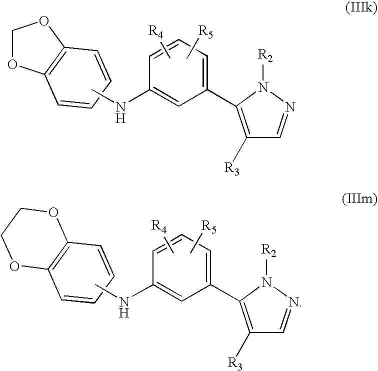

In some embodiments, compounds of Formulae (IIIe)-(IIIm) are optionally substituted with 1 to 3 substituents selected from the group consisting of C₁₋₅ acyl, C₁₋₄ alkoxy, C₁₋₆ alkyl, C₁₋₅ alkylcarboxamide, C₁₋₄ alkylsulfinyl, C₁₋₄ alkylsulfonyl, C₁₋₄ alkylthio, amino, C₁₋₄ alkylamino, C₂₋₅ dialkylamino, carboxamide, carboxy, carbo-C₁₋₆-alkoxy, cyano, C₂₋₈ dialkylcarboxamide, halogen, C₁₋₄ haloalkoxy, C₁₋₄ haloalkyl, hydroxyl, thiol, nitro and phenoxy; and where C₁₋₆ alkyl is optionally substituted with 1 to 3 substituents selected from the group consisting of C₁₋₄ alkoxy, C₁₋₅ alkylcarboxamide, amino, C₁₋₄ alkylamino, C₂₋₈ dialkylamino, halogen, C₁₋₄ haloalkoxy, C₁₋₄ haloalkyl, hydroxyl and thiol.

In some embodiments, compounds of the present invention are when R₁ is heteroaryl and is optionally substituted with 1 to 5 substituents selected independently from the group consisting of C₁₋₅ acyl, C₁₋₄ alkoxy, C₁₋₆ alkyl, C₁₋₅ alkylcarboxamide, C₁₋₄ alkylsulfinyl, C₁₋₄ alkylsulfonyl, C₁₋₄ alkylthio, amino, C₁₋₄ alkylamino, C₂₋₈ dialkylamino, carboxamide, carboxy, carbo-C₁₋₆-alkoxy, cyano, C₂₋₈ dialkylcarboxamide, halogen, C₁₋₄ haloalkoxy, C₁₋₄ haloalkyl, hydroxyl, thiol, nitro and phenoxy; and where C₁₋₆ alkyl is optionally substituted with 1 to 3 substituents selected from the group consisting of C₁₋₄ alkoxy, C₁₋₅ alkylcarboxamide, amino, C₁₋₄ alkylamino, C₂₋₈ dialkylamino, halogen, C₁₋₄ haloalkoxy, C₁₋₄ haloalkyl, hydroxyl and thiol. In some embodiments R₁ is heteroaryl and is optionally substituted with 1 to 3 substituents selected independently from the group consisting of C₁₋₄ alkoxy, C₁₋₆ alkyl, amino, C₁₋₄ alkylamino, C₂₋₈ dialkylamino, carbo-C₁₋₆-alkoxy, carboxamide, carboxy, halogen, C₁₋₄ haloalkoxy, C₁₋₄ haloalkyl, hydroxyl, thiol and nitro.

In some embodiments the heteroaryl is selected from the group consisting of quinolinyl, benzoxazolyl, benzimidazolyl, benzothiazolyl, quinazolinyl and pyrimidinyl as represented below:

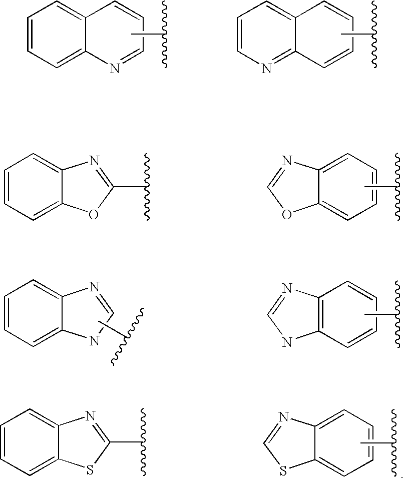

In some embodiments the heteroaryl is selected from the group consisting of benzoxazolyl-2-yl, quinolin-2-yl, quinolin-3-yl benzimidazol-2-yl, and benzothiazol-2-yl as represented below:

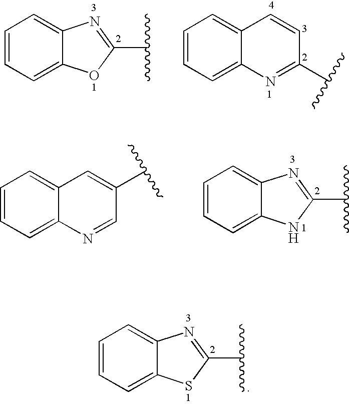

In some embodiments, compounds of the present invention are when R₂ is C₁₋₆ alkyl. In some embodiments R₂ is CH₃, CH₂CH₃, CH(CH₃)₂, or CH₂CH₂CH₃. In some embodiments R₂ is CH₃ and is represented by Formula (IIIn):

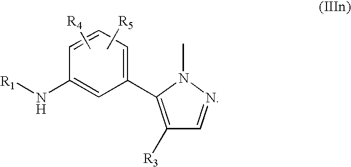

In some embodiments, compounds of the present invention are when R₃ is H, C₂₋₆ alkenyl, C₁₋₆ alkyl, C2-6 alkynyl, carbo-C₁₋₆-alkoxy, carboxamide, carboxy, cyano, C₃₋₇ cycloalkyl, halogen, 5 membered-heteroaryl or phenyl; and where C₂₋₆ alkenyl, C₁₋₆ alkyl or phenyl group may be optionally substituted with 1 to 3 substituents selected independently from the group consisting of C₁₋₄ alkoxy, C₂₋₆ alkynyl, C₂₋₈ dialkylamino, halogen, C₁₋₄ haloalkoxy and hydroxyl. In some embodiments R₃ is H, Cl, Br, CO₂CH₃, CO₂CH₂CH₃, 2-hydroxyethyl, 2-dimethylaminoethyl, 2-diethylaminoethyl, vinyl, CH₃, CH₂CH₃, phenyl, 4-methoxyphenyl, 3-methoxyphenyl, 4-fluorophenyl, 4-trifluoromthoxyphenyl, thiophenyl, CO₂H, cyclopropyl, —C≡CH, —CH═CH—CCH or CN. In some embodiments R₃ is H, Cl or Br.

Some embodiments of the present invention are compounds of Formula (I) wherein R₃ₐ is selected from the group consisting of H, C₁₋₆ alkyl and C₁₋₆ haloalkyl.

In some embodiments, R₃ₐ is selected from the group consisting of H, —CH₃, —CH₂CH₃, —CH(CH₃)₂, —CH₂CH₂CH₃, —CH₂CH(CH₃)₂, —CH₂CH₂CH₂CH₃, —CF₃, —CHF₂, —CFH₂, —CF₂CF₃ and —CH₂CF₃. In some embodiments, R₃ₐ is H or —CF₃. In some embodiments, R₃ₐ is H.

In some embodiments R₄ is H, halogen or NR₆R₇, wherein R₆R₇ is as defined herein. In some embodiments, R₄ is H, F, N(CH₃)₂, or pyrrolidin-1-yl. In some embodiments, R₄ is H.

In some embodiments, R₅ is halogen, or H. In some embodiments, R₅ is H.

In some embodiments, compounds of the present invention are represented by Formula (II): 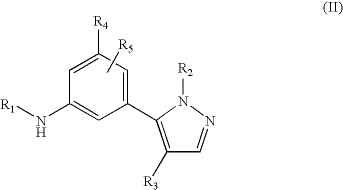
wherein:
   R₁, R₂ and R₃ have the same meaning as described herein;
   R₄ is H, C₁₋₄ alkoxy, phenyl, halogen, 5 or 6 membered-heteroaryl, hydroxyl, thiol or NR₆R₇, where the phenyl or heteroaryl group is optionally substituted with 1 to 5 substituents independently selected from the group consisting of C₁₋₅ acyl, C₁₋₄ alkoxy, C₁₋₆ alkyl, C₁₋₅ alkylcarboxamide, C₁₋₄ alkylsulfonyl, C₁₋₄ alkylthio, amino, C₁₋₄ alkylamino, C₂₋₈ dialkylamino, cyano, C₂₋₈ dialkylcarboxamide, halogen, C₁₋₄ haloalkoxy, C₁₋₄ haloalkyl, hydroxyl, thiol and nitro; and
   wherein:
      R₆ and R₇ are independently H, C₁₋₆ alkyl, or
      R₆ and R₇ together with the nitrogen to which they are bonded form a 5, 6 or 7 membered cyclic structure that may contain up to four heteroatoms selected from O, S or N—C₁₋₄ alkyl; and
   R₅ is H, C₁₋₄ alkoxy, C₁₋₆ alkyl, carboxamide, carboxy, cyano, halogen, C₁₋₄ haloalkoxy, C₁₋₄ haloalkyl, hydroxyl, thiol or nitro.

In some embodiments R₄ is NR₆R₇ wherein R₆ and R₇ together with the nitrogen to which they are bonded form a 5, 6 or 7 membered cyclic structure and may contain up to four heteroatoms selected from O, S or N—C₁₋₄ alkyl; these groups may be represented by the following Formulae:

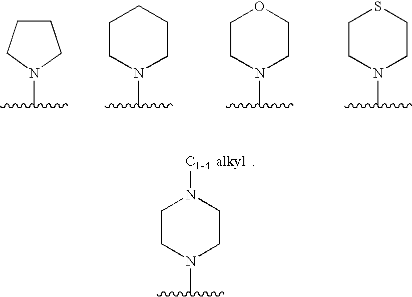

In some embodiments, compounds of the present invention are when R₄ is H, Cl, F, dimethylamino, diethylamino, pyrrolidin-1-yl, morpholin-1-yl, 4-methylpiperazin-1-yl, 4-ethylpiperazin-1-yl, hydroxyl, thiol, OCH₃ or OCH₂CH₃. In some embodiments R₅ is H or halogen.

In some embodiments, compounds of the present invention are when R₂ is CH₃. In some embodiments R₃ is H, Cl or Br.

In some embodiments R₄ is H, Cl, F, N(CH₃)₂ also referred to as dimethylamino, diethylamino, pyrrolidin-1-yl, morpholin-1-yl, 4-methylpiperazin-1-yl, 4-ethylpiperazin-1-yl, hydroxyl, thiol, OCH₃ or OCH₂CH₃.

One embodiment of the present invention includes compounds of Formula (IIIo): 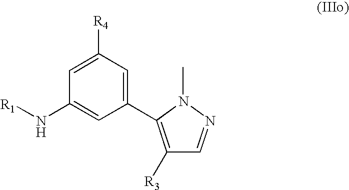
wherein:
   R₁ is phenyl substituted with 1 to 3 substituents selected independently from the group consisting of F, Cl, Br, I, CF₃, OCH₃ and OCF₃;
   R₃ is H, Cl or Br, and
   R₄ is H, Cl, F, dimethylamino, diethylamino, pyrrolidin-1-yl, morpholin-1-yl, 4-methylpiperazin-1-yl, 4-ethylpiperazin-1-yl, hydroxyl, thiol, OCH₃ or OCH₂CH₃; or
a pharmaceutically acceptable salt, hydrate or solvate thereof.

In some embodiments, compounds of the present invention are represented by Formula (IVa): 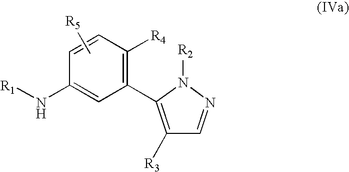
wherein R₁, R₂, R₃, R₄ and R₅ have the same meaning as described above.

In some embodiments, compounds have the Formula (IVa) wherein R₄ is H, C₁₋₄ alkoxy, C₁₋₆ alkyl, C₁₋₅ alkylcarboxamide, C₁₋₄ alkylsulfonamide, carbo-C₁₋₆-alkoxy, carboxamide, carboxy, cyano, C₂₋₈ dialkylcarboxamide, halogen, C₁₋₄ haloalkoxy, C₁₋₄ haloalkyl, hydroxyl, 5 or 6 membered-heteroaryl, phenyl or NR₆R₇, and wherein C₁₋₆ alkyl, 5 or 6 membered-heteroaryl or phenyl is optionally substituted with a substituents selected from the group consisting of C₁₋₅ acyl, C₁₋₅ acyloxy, C₂₋₆ alkenyl, C₁₋₄ alkoxy, C₁₋₅ alkyl, C₁₋₅ alkylcarboxamide, C₂₋₆ alkynyl, C₁₋₄ alkylsulfonamide, C₁₋₆ alkylureyl, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, hydroxyl, thiol and nitro;

wherein: R₆ and R₇ are independently C₁₋₆ alkyl optionally substituted with each R₆ and R₇ group may be optionally substituted with 1 to 5 substituents selected independently from the group consisting of H, C₁₋₅ acyl, C₂₋₆ alkenyl, C₁₋₄ alkoxy, C₁₋₆ alkyl, C₁₋₅ alkylcarboxamide, C₁₋₄ alkylthio, carbo-C₁₋₆-alkoxy, amino, C₁₋₄ alkylamino, C₂₋₈ dialkylamino, carboxamide, carboxamide, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro; or

R₆ and R₇ together with the nitrogen to which they are bonded form a 5, 6 or 7 membered cyclic structure which may be either saturated or unsaturated and that may contain up to four heteroatoms selected from O, NR₈ or S and the cyclic structure may be optionally substituted with 1 to 5 substituents selected independently from the group consisting of C₁₋₄ alkoxy, C₁₋₆ alkyl, C₁₋₅ alkylcarboxamide, carbo-C₁₋₆-alkoxy, amino, C₁₋₄ alkylamino, C₂₋₈ dialkylamino, carboxamide, carboxy, cyano, C₂₋₈ dialkylcarboxamide, halogen, C₁₋₄ haloalkoxy, C₁₋₄ haloalkyl, hydroxyl, thiol and nitro; and R₈ is H or C₁₋₆ alkyl.

In some embodiments, compounds have the Formula (IVa) wherein R₄ is H, C₁₋₄ alkoxy, C₁₋₆ alkyl, C₁₋₅ alkylcarboxamide, C₁₋₄ alkylsulfonamide, carbo-C₁₋₆-alkoxy, carboxamide, carboxy, cyano, C₂₋₈dialkylcarboxamide, halogen, C₁₋₄ haloalkoxy, C₁₋₄ haloalkyl, hydroxyl or NR₆R₇, and wherein C₁₋₆ alkyl is optionally substituted with a substituents selected from the group consisting of C₁₋₅ acyloxy, C₁₋₄ alkoxy, C₁₋₅ alkylcarboxamide, C₁₋₄ alkylsulfonamide, carbo-C₁₋₆-alkoxy, carboxamide, carboxy, C₂₋₈ dialkylcarboxamide, C₁₋₄ haloalkoxy, C₁₋₄ haloalkyl, hydroxyl, thiol and nitro;

wherein R₆ and R₇ are independently C₁₋₆ alkyl; or R₆ and R₇ together with the nitrogen to which they are bonded form a 5, 6 or 7 membered cyclic structure which is saturated and may contain up to four heteroatoms selected from O, NR₈ or S.

In some embodiments, compounds have the Formula (IVa) wherein R₄ is C₁₋₄ alkoxy, C₁₋₄ haloalkoxy, hydroxyl or NR₆R₇; wherein R₆ and R₇ are independently C₁₋₆ alkyl; or R₆ and R₇ together with the nitrogen to which they are bonded form a 5, 6 or 7 membered cyclic structure which is saturated and may contain one heteroatoms selected from O, NR₈ or S, where R₈ is CH₃ or CH₂CH₃.

In some embodiments, compounds have the Formula (IVa) wherein R₄ is OCH₃, OCH₂CH₃, OCH₂CH₂CH₃, OCH(CH₃)₂, OCH₂CH₂CH₂CH₃, OCH(CH₃)CH₂CH₃, OC(CH₃)₂CH₃ or OCH₂CH(CH₃)₂; R₁ is phenyl optionally substituted with 1 to 3 substituents selected independently from the group consisting of NO₂, F, Cl, Br, I, CF₃, OCH₃, OCF₃, SCH₃, S(O)CH₃, S(O)₂CH₃, CN, COCH₃, CH₃, CH₂OH and OC₆H₅; R₂ is C₁₋₆ alkyl and R₃ is H, Cl or Br. In some embodiments, R₁ is phenyl optionally substituted with 1 to 3 substituents selected independently from the group consisting of F, Cl, Br, I, CF₃, OCH₃ and OCF₃. In some embodiments, R₂ is CH₃ or CH₂CH₃. In some embodiments, R₂ is CH₃. In some embodiments, R₃ is Br.

In some embodiments, compounds have the Formula (IVa) wherein R₄ is OCH₃.

In some embodiments, compounds have the Formula (IVa) wherein R₄ is OCF₃, OCHF₂ or OCH₂CF₃; R₁ is phenyl optionally substituted with 1 to 3 substituents selected independently from the group consisting of NO₂, F, Cl, Br, I, CF₃, OCH₃, OCF₃, SCH₃, S(O)CH₃, S(O)₂CH₃, CN, COCH₃, CH₃, CH₂OH and OC₆H₅; R₂ is C₁₋₆ alkyl and R₃ is H, Cl or Br. In some embodiments, R₁ is phenyl optionally substituted with 1 to 3 substituents selected independently from the group consisting of F, Cl, Br, I, CF₃, OCH3 and OCF₃. In some embodiments, R₂ is CH₃ or CH₂CH₃. In some embodiments, R₂ is CH₃. In some embodiments, R₃ is Br.

In some embodiments, compounds have the Formula (IVa) wherein R₄ is OCF₃.

One embodiment of the present invention is the group of compounds wherein R₁ is phenyl substituted with at least one substituent selected from the group consisting of F, Cl, Br, I, CF₃, OCH₃ and OCF₃; R₂ is CH₃; and R₃, R₄ and R₅ are H.

One embodiment of the present invention is the group of compounds wherein R₁ is a phenyl substituted with at least one substituent selected from the group consisting of F, Cl, Br, I, CF₃, OCH₃ and OCF₃; R₂ is CH₃; R₂ is CH₃; R₂ is Cl; and both R₄ and R₅ are H.

One embodiment of the present invention is the group of compounds wherein R₁ is a phenyl substituted with at least one substituent selected from the group consisting of F, Cl, Br, I, CH₃, CF₃, OCH₃ and OCF₃; R₂ is CH₃; R₂ is CH₃; R₂ is Cl; and both R₄ and R₅ are H.

One embodiment of the present invention is the group of compounds wherein R₁ is a phenyl substituted with at least one substituent selected from the group consisting of F, Cl, Br, I, CH₃, CF₃, NO₂, C(O)CH₃, NHC(O)CH₃, CHOH, OC₆H₅, SCH₃, S(O)₂CH₃, CN, OCH₃ and OCF₃; R₂ is CH₃; R₂ is CH₃; R₂ is Br; and both R₄ and R₅ are H.

One embodiment of the present invention is the group of compounds wherein R₁ is a phenyl substituted with at least one substituent selected from the group consisting of F, Cl, Br, I, CH₃, CF₃, NO₂, C(O)CH₃, NHC(O)CH₃, CHOH, OC₆H₅, SCH₃, S(O)₂CH₃, CN, OCH₃ and OCF₃; or two substituents together with the phenyl form a methylenedioxy group (i.e., —OCH₂O—); R₂ is CH₃; R₂ is CH₃; R₂ is Br; and both R₄ and R₅ are H.

One embodiment of the present invention is the group of compounds wherein R₁ is a phenyl substituted with at least one substituent selected from the group consisting of F, Cl, CH₃, CF₃, CN, OCH₃ and OCF₃; R₂ is CH₃; R₂ is CH₃; R₂ is Br; and both R₄ and R₅ are H.

One embodiment of the present invention is the group of compounds wherein R₁ is a naphthyl optionally substituted with 1 to 3 substituents selected from the group consisting of F, Cl, CH₃, CF₃, CN, OCH₃ and OCF₃; R₂ is CH₃; R₂ is CH₃; R₂ is Br; and both R₄ and R₅ are H.

One embodiment of the present invention is the group of compounds wherein R₁ is a heteroaryl selected from benzoxazolyl-2-yl, quinolin-2-yl, quinolin-3-yl benzimidazol-2-yl, and benzothiazol-2-yl, and each heteroaryl optionally substituted with 1 to 3 substituents selected from the group consisting of F, Cl, CH₃, CF₃, CN, OCH₃ and OCF₃; R₂ is CH₃; R₂ is CH₃; R₂ is Br; and both R₄ and R₅ are H.

Some embodiments of the present invention include pharmaceutical compositions comprising compounds described herein and a pharmaceutically acceptable carrier.

Synthetic Methods for Making Compounds of the Invention:

The compounds of the present invention can be readily prepared according to a variety of synthetic regimes, all of which would be familiar to one skilled in the art. The chemical and patent literature quotes general procedures for the synthesis of intermediates and general compounds of Formula (I), one particular reference releated to the synthesis of certain R₄ substitutions and to phenyl-pyrazole coulpings is U.S. Provisional 60/401,467 filed Aug. 5, 2002 and is incorporated by reference in their entirety.

In the illustrated syntheses outlined below, the labeled substituents have the same identifications as set out in the definitions of the compound described above for Formula (I). The methods described below are useful in the preparation of compounds of the invention. Generally, compounds of Formula (I) were prepared by four separate methods and are labeled Methods A-D.

Method A:

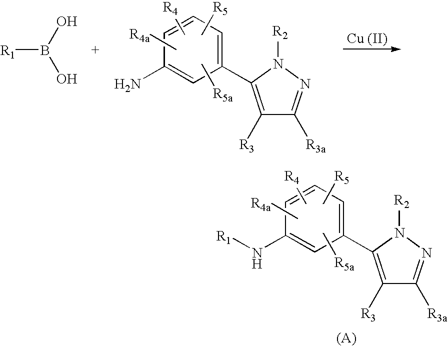

Method B:

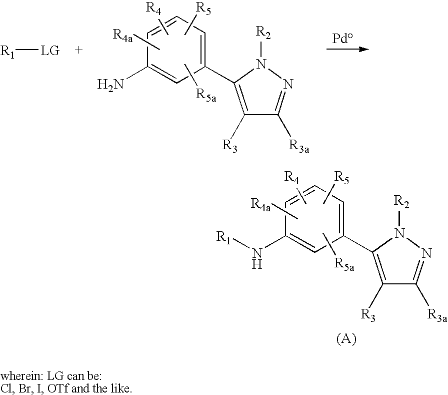

Method C:

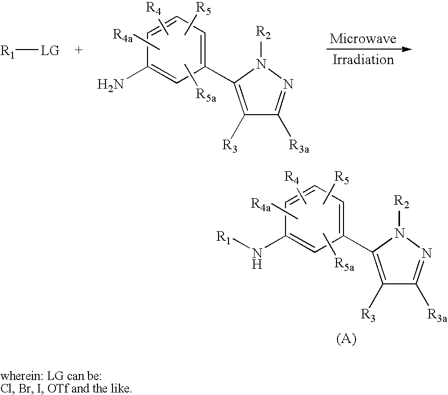

Irradiation with microwaves may be generated from a number of different microwaves sources. One particularly useful instrument in generating microwaves used in organic synthesis is the Smith Synthesizer and related instruments from Personal Chemistry AB, Uppsala Sweden.

Method D:

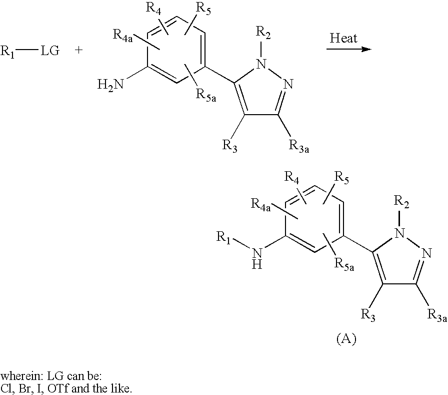

Additionally, compounds of Formula (I) encompass all pharmaceutically acceptable salts, solvates and particularly hydrates thereof The present invention also encompasses diastereomers as well as optical isomers, e.g. mixtures of enantiomers including racemic mixtures, as well as individual enantiomers and diastereomers, which arise as a consequence of structural asymmetry in certain compounds of Formula (I). Separation of the individual isomers or selective synthesis of the individual isomers is accomplished by application of various methods which are well known to practitioners in the art.

Certain examples of diarylamine and arylheteroarylamine pyrazole derivatives of Formula (I) are shown below in Table 2:

TABLE 2

| Compd | Chemical Structure | Chemical Name |
|---|---|---|
| 1 | 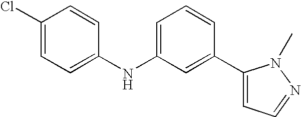 | (4-Chloro-phenyl)-[3-(2-methyl-2H-pyrazol-3- yl)-phenyl]-amine |
| 2 | 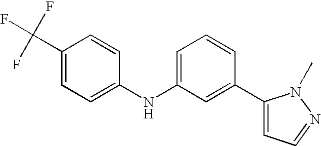 | [3-(2-Methyl-2H-pyrazol-3-yl)-phenyl]-(4- trifluoromethyl-phenyl)-amine |
| 3 | 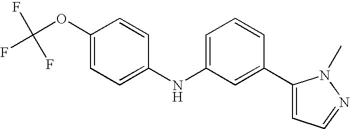 | [3-(2-Methyl-2H-pyrazol-3-yl)-phenyl]-(4- trifluoromethoxy-phenyl)-amine |
| 4 | 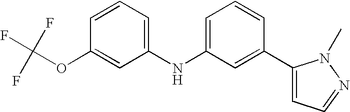 | [3-(2-Methyl-2H-pyrazol-3-yl)-phenyl]-(3- trifluoromethoxy-phenyl)-amine |
| 5 | 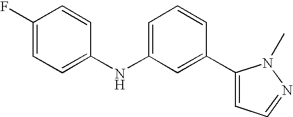 | [3-(2-Methyl-2H-pyrazol-3-yl)-phenyl]-(4- fluoro-phenyl)-amine |
| 6 | 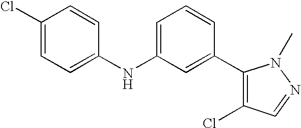 | [3-(4-Chloro-2-methyl-2H-pyrazol-3-yl)- phenyl]-(4-chloro-phenyl)-amine |
| 7 | 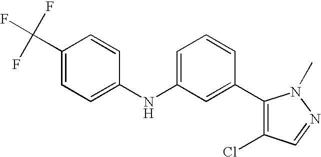 | [3-(4-Chloro-2-methyl-2H-pyrazol-3-yl)- phenyl]-(4-trifluoromethyl-phenyl)-amine |
| 8 | 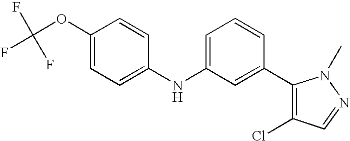 | [3-(4-Chloro-2-methyl-2H-pyrazol-3-yl)- phenyl]-(4-trifluoromethoxy-phenyl)-amine |
| 9 | 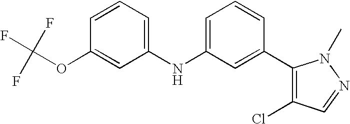 | [3-(4-Chloro-2-methyl-2H-pyrazol-3-yl)- phenyl]-(3-trifluoromethoxy-phenyl)-amine |
| 10 | 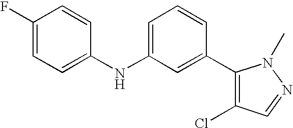 | [3-(4-Chloro-2-methyl-2H-pyrazol-3-yl)- phenyl]-(4-fluoro-phenyl)-amine |
| 11 | 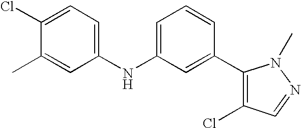 | [3-(4-Chloro-2-methyl-2H-pyrazol-3-yl)- phenyl]-(3-methyl-4-chloro-phenyl)-amine |
| 12 | 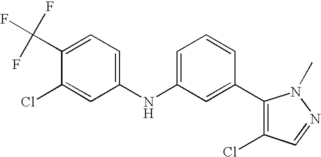 | [3-(4-Chloro-2-methyl-2H-pyrazol-3-yl)- phenyl]-(3-chloro-4-trifluoromethyl-phenyl)- amine |
| 13 | 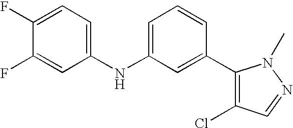 | [3-(4-Chloro-2-methyl-2H-pyrazol-3-yl)- phenyl]-(3,4-difluoro-phenyl)-amine |
| 14 | 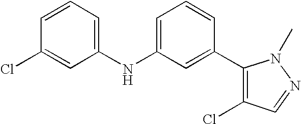 | [3-(4-Chloro-2-methyl-2H-pyrazol-3- yl)-phenyl]-(3-chloro-phenyl)-amine |
| 15 | 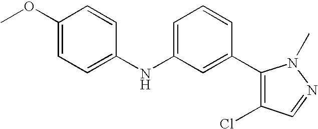 | [3-(4-Chloro-2-methyl-2H-pyrazol-3-yl)- phenyl]-(4-methoxy-phenyl)-amine |
| 16 | 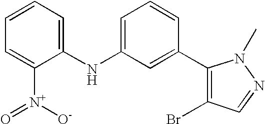 | [3-(4-Bromo-2-methyl-2H-pyrazol-3-yl)- phenyl]-(2-nitro-phenyl)-amine |
| 17 | 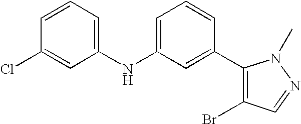 | [3-(4-Bromo-2-methyl-2H-pyrazol-3-yl)- phenyl]-(3-chloro-phenyl)-amine |
| 18 | 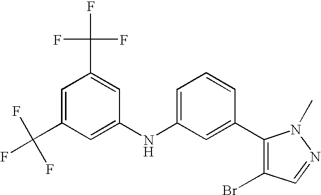 | [3-(4-Bromo-2-methyl-2H-pyrazol-3-yl)- phenyl]-(3,5-bis-trifluoromethyl-phenyl)-amine |
| 19 | 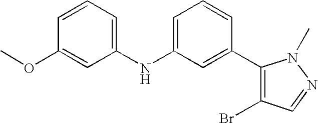 | [3-(4-Bromo-2-methyl-2H-pyrazol-3-yl)- phenyl]-(3-methoxy-phenyl)-amine |
| 20 | 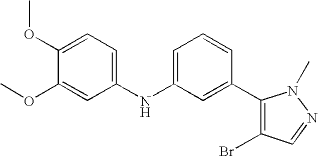 | [3-(4-Bromo-2-methyl-2H-pyrazol-3-yl)- phenyl]-(3,4-dimethoxy-phenyl)-amine |
| 21 | 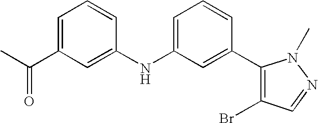 | 1-{3-[3-(4-Bromo-2-methyl-2H-pyrazol-3-yl)- phenylamino]-phenyl}-ethanone |
| 22 | 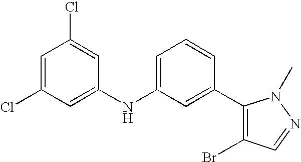 | [3-(4-Bromo-2-methyl-2H-pyrazol-3-yl)- phenyl]-(3,5-dichloro-phenyl)-amine |
| 23 | 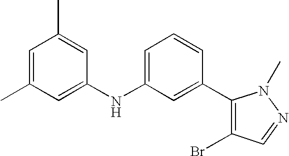 | [3-(4-Bromo-2-methyl-2H-pyrazol-3-yl)- phenyl]-(3,5-dimethyl-phenyl)-amine |
| 24 | 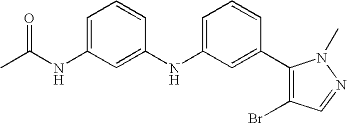 | N-{3-[3-(4-Bromo-2-methyl-2H-pyrazol-3-yl)- phenylamino]-phenyl}acetamide |
| 25 | 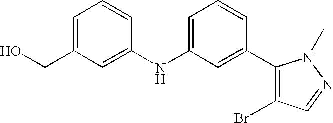 | {3-[3-(4-Bromo-2-methyl-2H-pyrazol-3-yl)- phenylamino]-phenyl}-methanol |
| 26 | 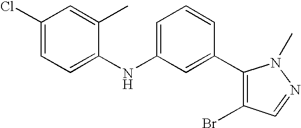 | [3-(4-Bromo-2-methyl-2H-pyrazol-3-yl)- phenyl]-(2-methyl-4-chloro-phenyl)-amine |
| 27 | 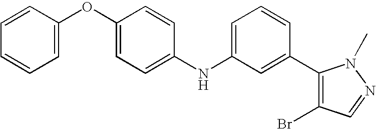 | [3-(4-Bromo-2-methyl-2H-pyrazol-3-yl)- phenyl]-(4-phenoxy-phenyl)-amine |
| 28 | 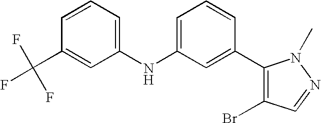 | [3-(4-Bromo-2-methyl-2H-pyrazol-3-yl)- phenyl]-(3-trifluoromethyl-phenyl)-amine |
| 29 | 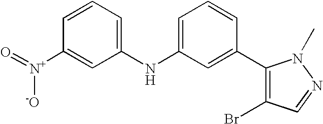 | [3-(4-Bromo-2-methyl-2H-pyrazol-3-yl)- phenyl]-(3-nitro-phenyl)-amine |
| 30 | 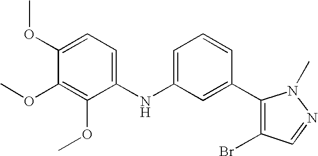 | [3-(4-Bromo-2-methyl-2H-pyrazol-3-yl)- phenyl]-(2,3,4-trimethoxy-phenyl)-amine |
| 31 | 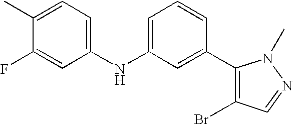 | [3-(4-Bromo-2-methyl-2H-pyrazol-3-yl)- phenyl]-(3-fluoro-4-methyl-phenyl)-amine |
| 32 | 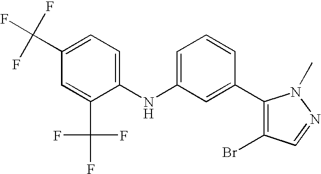 | [3-(4-Bromo-2-methyl-2H-pyrazol-3-yl)- phenyl]-(2,4-bis-trifluoromethyl-phenyl)-amine |
| 33 | 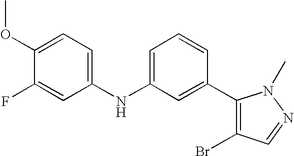 | [3-(4-Bromo-2-methyl-2H-pyrazol-3-yl)- phenyl]-(3-fluoro-4-methoxy-phenyl)-amine |
| 34 | 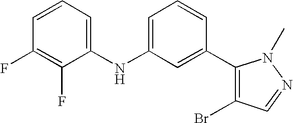 | [3-(4-Bromo-2-methyl-2H-pyrazol-3-yl)- phenyl]-(2,3-difluoro-phenyl)-amine |
| 35 | 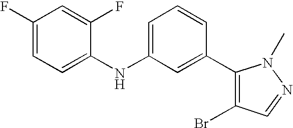 | [3-(4-Bromo-2-methyl-2H-pyrazol-3-yl)- phenyl]-(2,4-difluoro-phenyl)-amine |
| 36 | 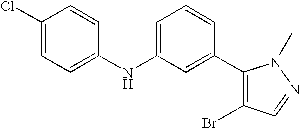 | [3-(4-Bromo-2-methyl-2H-pyrazo1-3-yl)- phenyl]-(4-chloro-phenyl)-amine |
| 37 | 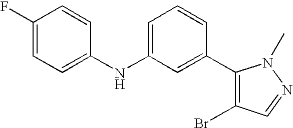 | [3-(4-Bromo-2-methyl-2H-pyrazol-3-yl)- phenyl]-(4-fluoro-phenyl)-amine |
| 38 | 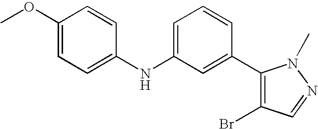 | [3-(4-Bromo-2-methyl-2H-pyrazol-3-yl)- phenyl]-(4-methoxy-phenyl)-amine |
| 39 | 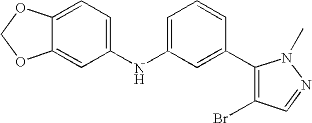 | Benzo[1,3]dioxol-5-yl-[3-(4-bromo-2-methyl- 2H-pyrazol-3-yl)-phenyl]-amine |
| 40 | 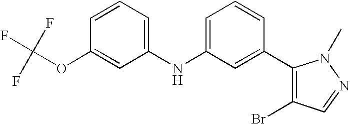 | [3-(4-Bromo-2-methyl-2H-pyrazol-3-yl)- phenyl]-(3-trifluoromethoxy-phenyl)-amine |
| 41 | 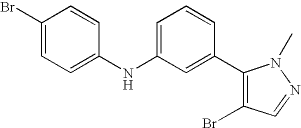 | [3-(4-Bromo-2-methyl-2H-pyrazol-3-yl)- phenyl]-(4-bromo-phenyl)-amine |
| 42 | 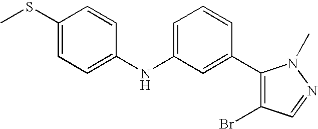 | [3-(4-Bromo-2-methyl-2H-pyrazol-3-yl)- phenyl]-(4-methylsulfanyl-phenyl)-amine |
| 43 | 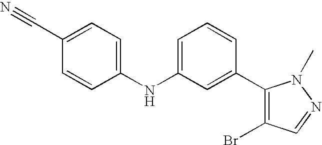 | 4-[3-(4-Bromo-2-methyl-2H-pyrazol-3-yl)- phenylamino]-benzonitrile |
| 44 | 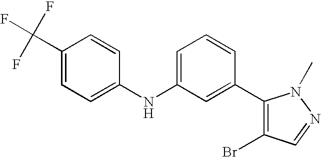 | [3-(4-Bromo-2-methyl-2H-pyrazol-3-yl)- phenyl]-(4-trifluoromethyl-phenyl)-amine |
| 45 | 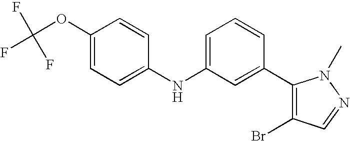 | [3-(4-Bromo-2-methyl-2H-pyrazol-3-yl)- phenyl]-(4-trifluoromethoxy-phenyl)-amine |
| 46 | 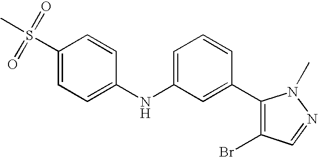 | [3-(4-Bromo-2-methyl-2H-pyrazol-3-yl)- phenyl]-(4-methanesulfonyl-phenyl)-amine |
| 47 | 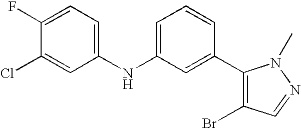 | [3-(4-Bromo-2-methyl-2H-pyrazol-3-yl)- phenyl]-(3-chloro-4-fluoro-phenyl)-amine |
| 48 | 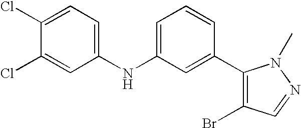 | [3-(4-Bromo-2-methyl-2H-pyrazol-3-yl)- phenyl]-(3,4-dichloro-phenyl)-amine |
| 49 | 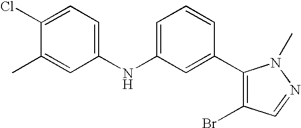 | [3-(4-Bromo-2-methyl-2H-pyrazol-3-yl)- phenyl]-(3-methyl-4-chloro-phenyl)-amine |
| 50 | 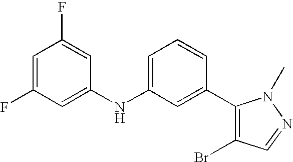 | [3-(4-Bromo-2-methyl-2H-pyrazol-3-yl)- phenyl]-(3,5-difluoro-phenyl)-amine |
| 51 | 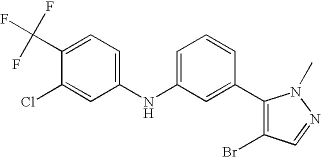 | [3-(4-Bromo-2-methyl-2H-pyrazol-3-yl)- phenyl]-(3-chloro-4-trifluoromethyl-phenyl)- amine |
| 52 | 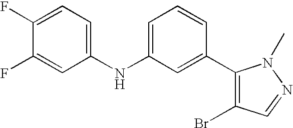 | [3-(4-Bromo-2-methyl-2H-pyrazol-3-yl)- phenyl]-(3,4-difluoro-phenyl)-amine |
| 53 | 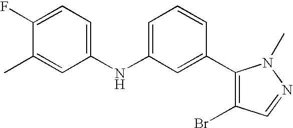 | [3-(4-Bromo-2-methyl-2H-pyrazol-3-yl)- phenyl]-(3-methyl-4-fluoro-phenyl)-amine |
| 54 | 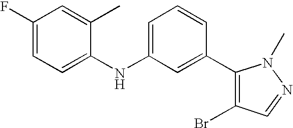 | [3-(4-Bromo-2-methyl-2H-pyrazol-3-yl)- phenyl]-(2-methyl-4-fluoro-phenyl)-amine |
| 55 | 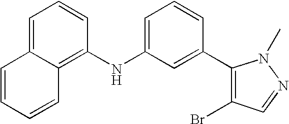 | [3-(4-Bromo-2-methyl-2H-pyrazol-3-yl)- phenyl]-naphthalen-1-yl-amine |
| 56 | 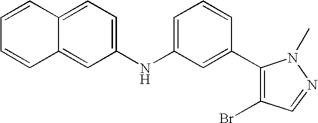 | [3-(4-Bromo-2-methyl-2H-pyrazol-3-yl)- phenyl]-naphthalen-2-yl-amine |
| 57 | 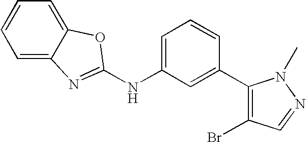 | Benzoxazol-2-yl-[3-(4-bromo-2-methyl-2H- pyrazol-3-yl)-phenyl]-amine |
| 58 | 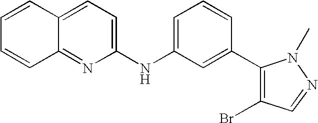 | [3-(4-Bromo-2-methyl-2H-pyrazol-3-yl)- phenyl]-quinolin-2-yl-amine |
| 59 | 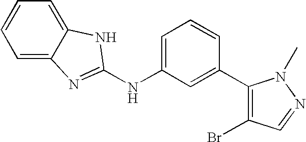 | (1H-Benzoimidazol-2-yl)-[3-(4-bromo-2- methyl-2H-pyrazol-3-yl)-phenyl]-amine |
| 60 | 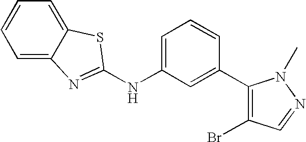 | Benzothiazol-2-yl-[3-(4-bromo-2-methyl-2H- pyrazol-3-yl)-phenyl]-amine |
| 61 | 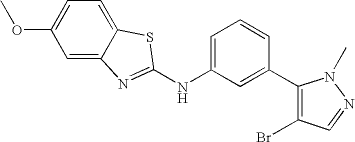 | [3-(4-Bromo-2-methyl-2H-pyrazol-3-yl)- phenyl]-(5-methoxy-benzothiazol-2-yl)-amine |
| 62 | 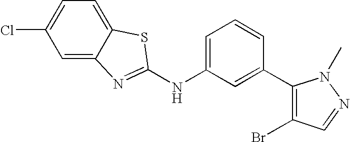 | [3-(4-Bromo-2-methyl-2H-pyrazol-3-yl)- phenyl]-(5-chloro-benzothiazol-2-yl)-amine |
| 63 | 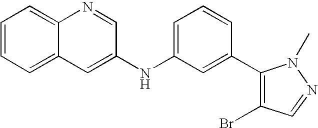 | [3-(4-Bromo-2-methyl-2H-pyrazol-3-yl)- phenyl]-quinolin-3-yl-amine |
| 64 | 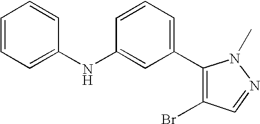 | [3-(4-Bromo-2-methyl-2H-pyrazol-3-yl)- phenyl]-phenyl-amine |
| 65 | 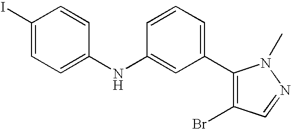 | [3-(4-Bromo-2-methyl-2H-pyrazol-3-yl)- phenyl]-(4-iodo-phenyl)-amine |
| 66 | 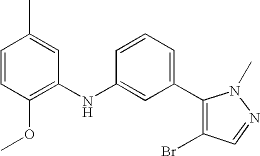 | [3-(4-Bromo-2-methyl-2H-pyrazol-3-yl)- phenyl]-(2-methoxy-5-methyl-phenyl)-amine |
| 67 | 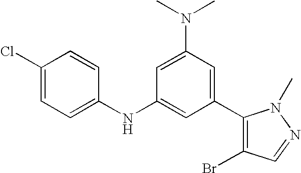 | [3-(4-Bromo-2-methyl-2H-pyrazol-3-yl)-5- (N,N-dimethylamino)-phenyl]-(4-chloro- phenyl)-amine |
| 68 | 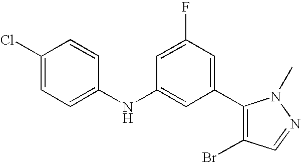 | [3-(4-Bromo-2-methyl-2H-pyrazol-3-yl)-5- fluoro-phenyl]-(4-chloro-phenyl)-amine |
| 69 | 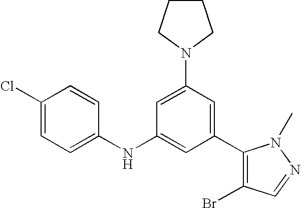 | [3-(4-Bromo-2-methyl-2H-pyrazol-3-yl)-5- pyrrolidin-1-yl-phenyl]-(4-chloro-phenyl)- amine |
| 70 | 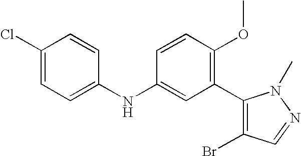 | [3-(4-Bromo-2-methyl-2H-pyrazol-3-yl)-4- methoxy-phenyl)-(4-chloro-phenyl)-amine |
| 71 | 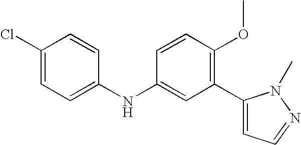 | (4-Chloro-phenyl)-[4-methoxy-3-(2-methyl-2H- pyrazol-3-yl)-phenyl]-amine |
| 72 | 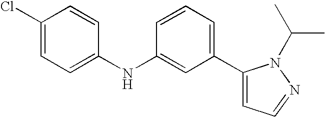 | (4-Chloro-phenyl)-[3-(2-isopropyl-2H-pyrazol- 3-yl)-phenyl]-amine |
| 73 | 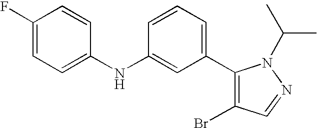 | [3-(4-Bromo-2-isopropyl-2H-pyrazol-3-yl)- phenyl]-(4-fluoro-phenyl)-amine |
| 74 | 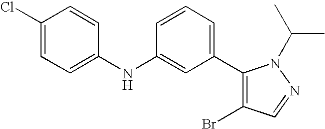 | [3-(4-Bromo-2-isopropyl-2H-pyrazol-3-yl)- phenyl]-(4-chloro-phenyl)-amine |
| 75 | 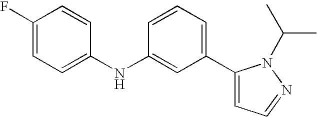 | (4-Fluoro-phenyl)-[3-(2-isopropyl-2H-pyrazol- 3-yl)-phenyl]-amine |
| 76 | 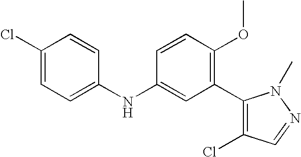 | [3-(4-Chloro-2-methyl-2H-pyrazol-3-yl)-4- methoxy-phenyl]-(4-chloro-phenyl)-amine |
| 77 | 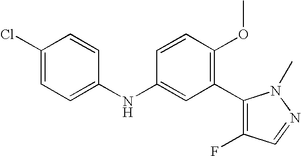 | (4-Chloro-phenyl)-[3-(4-fluoro-2-methyl-2H- pyrazol-3-yl)-4-methoxy-phenyl]-amine |
| 78 | 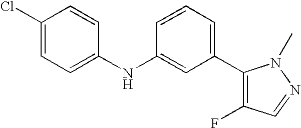 | (4-Chloro-phenyl)-[3-(4-fluoro-2-methyl-2H- pyrazol-3-yl)-phenyl]-amine |

In addition to the foregoing beneficial uses for the modulators of 5HT_{2A} receptor activity disclosed herein, the compounds disclosed herein are useful in the treatment of one or more additional diseases and disorders, and in the amelioration of symptoms thereof. Without limitation, these include the following:

1. Antiplatelet Therapies (5HT_{2A} Mediated Platelet Aggregation):

Antiplatelet agents (antiplatelets) are prescribed for a variety of conditions. For example, in coronary artery disease they are used to help prevent myocardial infarction or stroke in patients who are at risk of developing obstructive blood clots (e.g., coronary thrombosis).

In a myocardial infarction (heart attack), the heart muscle does not receive enough oxygen-rich blood as a result of a blockage in the coronary blood vessels. If taken while an attack is in progress or immediately afterward (preferably within 30 minutes), antiplatelets can reduce the damage to the heart.

A transient ischemic attack (“TIA” or “mini-stroke”) is a brief interruption of oxygen flow to the brain due to decreased blood flow through arteries, usually due to an obstructing blood clot. Antiplatelet drugs have been found to be effective in preventing TIAs.

Angina is a temporary and often recurring chest pain, pressure or discomfort caused by inadequate oxygen-rich blood flow (ischemia) to some parts of the heart. In patients with angina, antiplatelet therapy can reduce the effects of angina and the risk of myocardial infarction.

Stroke is an event in which the brain does not receive enough oxygen-rich blood, usually due to blockage of a cerebral blood vessel by a blood clot. In high-risk patients, taking antiplatelets regularly has been found to prevent the formation blood clots that cause first or second strokes.

Angioplasty is a catheter based technique used to open arteries obstructed by a blood clot. Whether or not stenting is performed immediately after this procedure to keep the artery open, antiplatelets can reduce the risk of forming additional blood clots following the procedure(s).

Coronary bypass surgery is a surgical procedure in which an artery or vein is taken from elsewhere in the body and grafted to a blocked coronary artery, rerouting blood around the blockage and through the newly attached vessel. After the procedure, antiplatelets can reduce the risk of secondary blood clots.

Atrial fibrillation is the most common type of sustained irregular heart rhythm (arrythmia). Atrial fibrillation affects about two million Americans every year. In atrial fibrillation, the atria (the heart's upper chambers) rapidly fire electrical signals that cause them to quiver rather than contract normally. The result is an abnormally fast and highly irregular heartbeat. When given after an episode of atrial fibrillation, antiplatelets can reduce the risk of blood clots forming in the heart and traveling to the brain (embolism).

5HT_{2A} receptors are expressed on smooth muscle of blood vessels and 5HT secreted by activated platelets causes vasoconstriction as well as activation of additional platelets during clotting. There is evidence that a 5HT_{2A} inverse agonist will inhibit platelet aggregation and thus be a potential treatment as an antiplatelet therapy. See Satimura, K, et al., Clin Cardiol 2002 Jan. 25 (1):28-32; and Wilson, H. C et al., Thromb Haemost Sep. 2, 1991 ;66(3):355-60.

The 5HT_{2A} inverse agonists disclosed herein provide beneficial improvement in microcirculation to patients in need of antiplatelet therapy by antagonizing the vasoconstrictive products of the aggregating platelets in, for example and not limitation, the indications described above. Accordingly, in some embodiments, the present invention provides methods for reducing platelet aggregation in an individual in need thereof comprising administering to the individual a composition comprising a 5HT_{2A} inverse agonist disclosed herein. In further embodiments, the present invention provides methods for treating coronary artery disease, myocardial infarction, transient ischemic attack, angina, stroke, atrial fibrillation, or a symptom of any of the foregoing in an individual in need of said treatment, comprising administering to the individual a composition comprising a 5HT_{2A} inverse agonist disclosed herein.

In further embodiments, the present invention provides methods for reducing risk of blood clot formation in a angioplasty or coronary bypass surgery individual, or an individual suffering from atrial fibrillation, comprising administering to the individual a composition comprising a 5HT_{2A} inverse agonist disclosed herein at a time where such risk exists.

2. Asthma

It has been suggested that 5-HT (5-hydroxytryptamine) plays a role in the pathophysiology of acute asthma. See Cazzola, M. and Matera, M. G., TiPS, 2000, 21, 13; and De Bie, J. J. et al., British J. Pharm, 1998, 124, 857-864. The compounds of the present invention disclosed herein are useful in the treatment of asthma, and the treatment of the symptoms thereof. Accordingly, in some embodiments, the present invention provides methods for treating asthma in an individual in need of said treatment, comprising administering to the individual a composition comprising a 5HT_{2A} inverse agonist disclosed herein. In further embodiments, methods are provided for treating a symptom of asthma in an individual in need of said treatment, comprising administering to the individual a composition comprising a 5RT_{2A} inverse agonist disclosed herein.

3. Agitation

Agitation is a well-recognized behavioral syndrome with a range of symptoms, including hostility, extreme excitement, poor impulse control, tension and uncooperativeness (See Cohen-Mansfield J, and Billig, N., (1986), Agitated Behaviors in the-Elderly. I. A Conceptual Review. J Am Geriatr Soc 34(10): 711-721).

Agitation is a common occurrence in the elderly and often associated with dementia such as those caused by Alzheimers's disease, Lewy Body, Parkinson's, and Huntington's, which are degenerative diseases of the nervous system and by diseases that affect blood vessels, such as stroke, or multi-infarct dementia, which is caused by multiple strokes in the brain can also induce dementia. Alzheimer's disease accounts for approximately 50 to 70% of all dementias (See Koss E, et al., (1997), Assessing patterns of agitation in Alzheimer's disease patients with the Cohen-Mansfield Agitation Inventory. The Alzheimer's Disease Cooperative Study. Alzheimer Dis Assoc Disord 11(suppl 2):S45-S50).

An estimated five percent of people aged 65 and older and up to 20 percent of those aged 80 and older are affected by dementia. Of these sufferers, nearly half exhibit behavioral disturbances, such as agitation, wandering and violent outbursts.

Agitated behaviors can also be manifested in cognitively intact elderly people and by those with psychiatric disorders other than dementia

Agitation is often treated with antipsychotic medications such as haloperidol in nursing home and other assisted care settings. There is emerging evidence that agents acting at the 5HT_{2A} receptors in the brain have the effects of reducing agitation in patients, including Alzheimer's dementia (See Katz, I. R., et al., J Clin Psychiatry February 1999, 60(2): 107-115; and Street, J. S., et al., Arch Gen Psychiatry October 2000, 57(10):968-976).

The compounds of the invention disclosed herein are useful for treating agitation and symptoms thereof. Thus, in some embodiments, the present invention provides methods for treating agitation in an individual in need of such treatment comprising administering to the individual a composition comprising a 5HT_{2A} inverse agonist disclosed herein. In some embodiments, the agitation is due to a psychiatric disorder other than dementia. In some embodiments, the present invention provides methods for treatment of agitation or a symptom thereof in an individual suffering from dementia comprising administering to the individual a composition comprising a 5HT_{2A} inverse agonist disclosed herein. In some embodiments of such methods, the dementia is due to a degenerative disease of the nervous system, for example and without limitation, Alzheimers disease, Lewy Body, Parkinson's disease, and Huntington's disease, or dementia due to diseases that affect blood vessels, including with out limitation stroke and multi-infarct dementia. In some embodiments, methods are provided for treating agitation or a symptom thereof in an individual in need of such treatment, where the individual is a cognitively intact elderly patient, comprising administering to the individual a composition comprising a 5HT_{2A} inverse agonist disclosed herein.

4. Add-On Therapy to Haloperidol in the Treatment of Schizophrenia and Other Disorders:

Schizophrenia is a psychopathic disorder of unknown origin, which usually appears for the first time in early adulthood and is marked by a number of characteristics, psychotic symptoms, progression, phasic development and deterioration in social behavior and professional capability in the region below the highest level ever attained. Characteristic psychotic symptoms are disorders of thought content (multiple, fragmentary, incoherent, implausible or simply delusional contents or ideas of persecution) and of mentality (Goss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook).

Haloperidol (Haldol) is a potent dopamine D2 receptor antagonist. It is widely prescribed for acute schizophrenic symptoms, and is very effective for the positive symptoms of schizophrenia. However, Haldol is not effective for the negative symptoms of schizophrenia and may actually induce negative symptoms as well as cognitive dysfunction. In accordance with some methods of the invention, adding a 5HT_{2A} inverse agonist concomitantly with Haldol will provide benefits including the ability to use a lower dose of Haldol without losing its effects on positive symptoms, while reducing or eliminating its inductive effects on negative symptoms, and prolonging relapse to the individual's next schizophrenic event.

Haloperidol is used for treatment of a variety of behavioral disorders, drug induced psychosis, excitative psychosis, Gilles de la Tourette's syndrome, manic disorders, psychosis (organic and NOS), psychotic disorder, psychosis, schizophrenia (acute, chronic and NOS). Further uses include in the treatment of infantile autism, Huntington's chorea, and nausea and vomiting from chemotherapy and chemotherapeutic antibodies. Administration of 5HT_{2A} inverse agonists disclosed herein with haloperidol also will provide benefits in these indications.

In some embodiments, the present invention provides methods for treating a behavioral disorder, drug induced psychosis, excitative psychosis, Gilles de la Tourette's syndrome, manic disorders, psychosis (organic and NOS), psychotic disorder, psychosis, schizophrenia (acute, chronic and NOS) comprising administering to the individual a dopamine D2 receptor antagonist and a 5HT_{2A} inverse agonist disclosed herein.

In some embodiments, the present invention provides methods for treating a behavioral disorder, drug induced psychosis, excitative psychosis, Gilles de la Tourette's syndrome, manic disorders, psychosis (organic and NOS), psychotic disorder, psychosis, schizophrenia (acute, chronic and NOS) comprising administering to the individual haloperidol and a 5HT_{2A} inverse agonist disclosed herein.

In some embodiments, the present invention provides methods for treating infantile autism, Huntington's chorea, or nausea and vomiting from chemotherapy or chemotherapeutic antibodies comprising administering to the individual a dopamine D2 receptor antagonist and a 5HT_{2A} inverse agonist disclosed herein.

In some embodiments, the present invention provides methods for treating infantile autism, Huntington's chorea, or nausea and vomiting from chemotherapy or chemotherapeutic antibodies comprising administering to the individual haloperidol and a 5HT_{2A} inverse agonist disclosed herein.

In further embodiments, the present invention provides methods for treating schizophrenia in an individual in need of said treatment comprising administering to the individual a dopamine D2 receptor antagonist and a 5HT_{2A} inverse agonist disclosed herein. Preferably, the dopamine D2 receptor antagonist is haloperidol.

The administration of the dopamine D2 receptor antagonist can be concomitant with administration of the 5HT_{2A} inverse agonist, or they can be administered at different times. Those of skill in the art will easily be able to determine appropriate dosing regimes for the most efficacious reduction or elimination of deleterions haloperidol effects. In some embodiments, haloperidol and the 5HT_{2A} inverse agonist are administered in a single dosage form, and in other embodiments, they are administered in separate dosage forms.

The present invention further provides methods of alleviating negative symptoms of schizophrenia induced by the administration of haloperidol to an individual suffering from said schizophrenia, comprising administering to the individual a 5HT_{2A} inverse agonist as disclosed herein.

5. Sleep Disorders

It is reported in the National Sleep Foundation's 2002 Sleep In America Poll, more than one-half of the adults surveyed (58%) report having experienced one or more symptoms of insomnia at least a few nights a week in the past year. Additionally, about three in ten (35%) say they have experienced insomnia-like symptoms every night or almost every night.

The normal sleep cycle and sleep architecture can be disrupted by a variety of organic causes as well as environmental influences. According to the International Classification of Sleep Disorders, there are over 80 recognized sleep disorders. Of these, compounds of Formula (I) are effective, for example, in any one or more of the following sleep disorders (ICSD—International Classification of Sleep Disorders: Diagnostic and Coding Manual. Diagnostic Classification Steering Committee, American Sleep Disorders Association, 1990):

A. Dyssomnias

a. Intrinsic Sleep Disorders:

Psychophysiological insomnia, Sleep state misperception, Idiopathic insomnia, Obstructive sleep apnea syndrome, Central sleep apnea syndrome, Central alveolar hypoventilation syndrome, Periodic limb movement disorder, Restless leg syndrome and Intrinsic sleep disorder NOS.

b. Extrinsic Sleep Disorders:

Inadequate sleep hygiene, Environmental sleep disorder, Altitude insomnia, Adjustment sleep disorder, Insufficient sleep syndrome, Limit-setting sleep disorder, Sleep-onset association disorder, Nocturnal eating (drinking) syndrome, Hypnotic dependent sleep disorder, Stimulant-dependent sleep disorder, Alcohol-dependent sleep disorder, Toxin-induced sleep disorder and Extrinsic sleep disorder NOS.

c. Circadian Rhythm Sleep Disorders:

Time zone change (jet lag) syndrome, Shift work sleep disorder, Irregular sleep-wake pattern, Delayed sleep phase syndrome, Advanced sleep phase syndrome, Non-24hour sleep-wake disorder and Circadian rhythm sleep disorder NOS.

B. Parasomnias

a. Arousal Disorders:

Confusional arousals, Sleepwalking and Sleep terrors.

b. Sleep-Wake Transition Disorders:

Rhythmic movement disorder, Sleep starts, Sleep talking and Nocturnal leg cramps.

C. Sleep Disorders Associated with Medical/Psychiatric Disorders

a. Associated with Mental Disorders:

Psychoses, Mood disorders, Anxiety disorders, Panic disorders and Alcoholism.

b. Associated with Neurological Disorders:

Cerebral degenerative disorders, Dementia, Parkinsonism, Fatal familial insomnia, Sleep-related epilepsy, Electrical status epilepticus of sleep and Sleep-related headaches.

c. Associated with Other Medical Disorders:

Sleeping sickness, Nocturnal cardiac ischemia, Chronic obstructive pulmonary disease Sleep-related asthma, Sleep-related gastroesophageal reflux, Peptic ulcer disease, Fibrositis syndrome, Osteoarthritis, Rheumatoid arthritis, Fibromyalgia and Post-surgical.

The effects of sleep deprivation are more than excessive daytime sleepiness. Chronic insomniacs report elevated levels of stress, anxiety, depression and medical illnesses (National Institutes of Health, National Heart, Lung, and Blood Institute, Insomnia Facts Sheet, October 1995). Preliminary evidence suggests that having a sleep disorder that causes significant loss of sleep may contribute to increased susceptibility to infections due to immunosuppression, cardiovascular complications such as hypertension, cardiac arrhythmias, stroke, and myocardial infarction. Compounds of Formula (I) are useful to prevent or alleviate these complications by improving sleep quality.

The most common class of medications for the majority of sleep disorders are the benzodiazepines, but the adverse effect profile of benzodiazepines include daytime sedation, diminished motor coordination, and cognitive impairments. Furthermore, the National Institutes of Health Consensus conference on Sleeping Pills and Insomnia in 1984 have developed guidelines discouraging the use of such sedative-hypnotics beyond 4-6 weeks because of concerns raised over drug misuse, dependency, withdrawal and rebound insomnia. Therefore, it is desirable to have a pharmacological agent for the treatment of insomnia, which is more effective and/or has fewer side effects that those currently used.

Clinical studies with agents of a similar mechanism of action as are compounds of Formula (I) have demonstrated significant improvements on objective and subjective sleep parameters in normal, healthy volunteers as well as patients with sleep disorders and mood disorders [Sharpely AL, et al. Slow Wave Sleep in Humans: Role of 5HT_{2A} and 5HT2c Receptors. Neuropharmacology, 1994, Vol. 33(3/4):467-71; Winokur A, et al. Acute Effects of Mirtazapine on Sleep Continuity and Sleep Architecture in Depressed Patients: A Pilot Study. Soc of Biol Psych, 2000, Vol. 48:75-78; and Landolt HP, et al. Serotonin-2 Receptors and Human Sleep: Effect of Selective Antagonist on EEG Power Spectra. Neuropsychopharmacology, 1999, Vol. 21(3):455-66].

Some sleep disorders are sometimes found in conjunction with other conditions and accordingly those conditions are treatable by compounds of Formula (I). For example but not limiting, patients suffering from mood disorders typically suffer from a sleep disorder that can be treatable by compounds of Formula (I). Having one pharmacological agent which treats two or more existing or potential conditions, as does the present invention, is more cost effective, leads to better compliance and has fewer side effects than taking two or more agents.

It is an object of the present invention to provide a therapeutic agent for the use in treating Sleep Disorders. It is another object of the present invention to provide one pharmaceutical agent, which may be useful in treating two or more conditions wherein one of the conditions is a sleep disorder. Compounds of Formula (I) described herein may be used alone or in combination with a mild sleep inducer (i.e. antihistamine).

Sleep Architecture

Sleep comprises two physiological states: Non rapid eye movement (NREM) and rapid eye movement (REM) sleep. NREM sleep consists of four stages, each of which is characterized by progressively slower brain wave patterns, with the slower patterns indicating deeper sleep. So-called delta sleep, stages 3 and 4 of NREM sleep, is the deepest and most refreshing type of sleep. Many patients with sleep disorders are unable to adequately achieve the restorative sleep of stages 3 and 4. In clinical terms, patients' sleep patterns are described as fragmented, meaning the patient spends a lot of time alternating between stages 1 and 2 (semi-wakefulness) and being awake and very little time in deep sleep. Compounds of Formula (I) described are effective in consolidating sleep patterns so that the patient with previously fragmented sleep can now achieve restorative, delta-wave sleep for longer, more consistent periods of time.

As sleep moves from stage 1 into later stages, heart rate and blood pressure drop, metabolic rate and glucose consumption fall, and muscles relax. NREM sleep makes up about 75% of total sleep time; stage 1 accounting for 5-10% of total sleep time, stage 2 for about 45-50%, stage 3 approximately 12%, and stage 4 13-15%. About 90 minutes after sleep onset, NREM sleep gives way to the first REM sleep episode of the night. REM makes up approximately 25% of total sleep time. In contrast to NREM sleep, REM sleep is characterized by high pulse, respiration, and blood pressure, as well as other physiological patterns similar to those seen in the active waking stage. Hence, REM sleep is also known as “paradoxical sleep.” Sleep onset usually occurs during NREM sleep and takes 10-20 minutes in healthy young adults. The four stages of NREM sleep together with a REM phase form one complete sleep cycle that is repeated throughout the duration of sleep, usually four or five times. The cyclical nature of sleep is regular and reliable; a REM period occurs about every 90 minutes during the night. However, the first REM period tends to be the shortest, often lasting less than 10 minutes, whereas the later REM periods may last up to 40 minutes. With aging, the time between retiring and sleep onset increases and the total amount of night-time sleep decreases because of changes in sleep architecture that impair sleep maintenance as well as sleep quality. Both NREM (particularly stages 3 and 4) and REM sleep are reduced. However, stage 1 NREM sleep, which is the lightest sleep, increases with age.

Subjective and Objective Determinations of Sleep Disorders

There are a number of ways to determine whether the onset, duration or quality of sleep (e.g. non-restorative or restorative sleep) is impaired or improved. One method is a subjective determination of the patient, e.g., do they feel drowsy or rested upon waking. Other methods involve the observation of the patient by another during sleep, e.g., how long it takes the patient to fall asleep, how many times does the patient wake up during the night, how restless is the patient during sleep, etc. Another method is to objectively measure the stages of sleep.

Polysomnography is the monitoring of multiple electrophysiological parameters during sleep and generally includes measurement of EEG activity, electroculographic activity and electromyographic activity, as well as other measurements. These results, along with observations, can measure not only sleep latency (the amount of time required to fall asleep), but also sleep continuity (overall balance of sleep and wakefulness) and sleep consolidation (percent of sleeping time spent in delta-wave or restorative sleep) which may be an indication of the quality of sleep.

There are five distinct sleep stages, which can be measured by polysomnography: rapid eye movement (REM) sleep and four stages of non-rapid eye movement (NREM) sleep (stages 1, 2, 3 and 4). Stage 1 NREM sleep is a transition from wakefulness to sleep and occupies about 5% of time spent asleep in healthy adults. Stage 2 NREM sleep, which is characterized by specific EEG waveforms (sleep spindles and K complexes), occupies about 50% of time spent asleep. Stages 3 and 4 NREM sleep (also known collectively as slow-wave sleep and delta-wave sleep) are the deepest levels of sleep and occupy about 10-20% of sleep time. REM sleep, during which the majority of vivid dreams occur, occupies about 20-25% of total sleep.

These sleep stages have a characteristic temporal organization across the night. NREM stages 3 and 4 tend to occur in the first one-third to one-half of the night and increase in duration in response to sleep deprivation. REM sleep occurs cyclically through the night Alternating with NREM sleep about every 80-100 minutes. REM sleep periods increase in duration toward the morning. Human sleep also varies characteristically across the life span. After relative stability with large amounts of slow-wave sleep in childhood and early adolescence, sleep continuity and depth deteriorate across the adult age range. This deterioration is reflected by increased wakefulness and stage 1 sleep and decreased stages 3 and 4 sleep.

Accordingly, another aspect of the present invention relates to the therapeutic use of compounds of Formula (I) for the treatment of Sleep Disorders. Compounds of Formula (I) are potent inverse agonists at the serotonin 5HT₂ receptors and are effective in the treatment of Sleep Disorders by promoting one or more of the following: reducing the sleep onset latency period (measure of sleep induction), reducing the number of nighttime awakenings, and prolonging the amount of time in delta-wave sleep (measure of sleep quality enhancement) without effecting REM sleep. In addition, compounds of Formula (I) are effective either as a monotherapy or in combination with sleep inducing agents, for example but not limiting, antihistamines.

Pharmaceutical Compositions

Suitable pharmaceutically-acceptable carriers are available to those in the art; for example, see Remington: The Science and Practice or Pharmacy, 20^{th} Edition, 2000, Lippincott, Williams & Wilkons, (Gennaro et al., eds.).

While it is possible that, for use in the prophylaxis or treatment, a compound of the invention may in an alternative use be administered as a raw or pure chemical, it is preferable however to present the compound or active ingredient as a pharmaceutical formulation or composition.

The invention thus further provides pharmaceutical formulations comprising a compound of the invention or a pharmaceutically acceptable salt or derivative thereof together with one or more pharmaceutically acceptable carriers therefor and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The dose when using the compounds of Formula (I) can vary within wide limits, and as is customary and is known to the physician, it is to be tailored to the individual conditions in each individual case. It depends, for example, on the nature and severity of the illness to be treated, on the condition of the individual, on the compound employed or on whether an acute or chronic disease state is treated or prophylaxis is conducted or on whether further active compounds are administered in addition to the compounds of the Formula (I). Representative doses of the present invention include, about 0.01 mg to about 1000 mg, about 0.01 to about 750 mg, about 0.01 to about 500 mg, 0.01 to about 250 mg, 0.01 mg to about 200 mg, about 0.01 mg to 150 mg, about 0.01 mg to about 100 mg, and about 0.01 mg to about 75 mg. Multiple doses may be administered during the day, especially when relatively large amounts are deemed to be needed, for example 2, 3 or 4, doses. If appropriate, depending on individual behavior and as appropriate from the individuals physician or care-giver it may be necessary to deviate upward or downward from the daily dose.

The amount of active ingredient, or an active salt or derivative thereof, required for use in treatment will vary not only with the particular salt selected but also with the route of administration, the nature of the condition being treated and the age and condition of the individual and will ultimately be at the discretion of the attendant physician or clinician. In general, one skilled in the art understands how to extrapolate in vivo data obtained in a model system, typically an animal model, to another, such as a human. An illustrative but not intended to be limiting in vivo animal model is provided as an Example infra. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the individual, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the Formula (I) and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate or hydrate of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets include, for example, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the individual administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the Formula (I) or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the Formula (I) as an aerosol can be prepared by processes well-known to the person skilled in the art. For their preparation, for example, solutions or dispersions of the compounds of the Formula (I) in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

### EXAMPLES

The following Examples are provided for illustrative purposes and not as a means of limitation.

### Example 1

### Method A: Synthesis of Compound 36, [3-(4-Bromo-2-methyl-2H-pyrazol-3-yl)-phenyl]-(4-chloro-phenyl)-amine, using Copper (II) Acetate and 4-Chlorophenyl Boronic acid

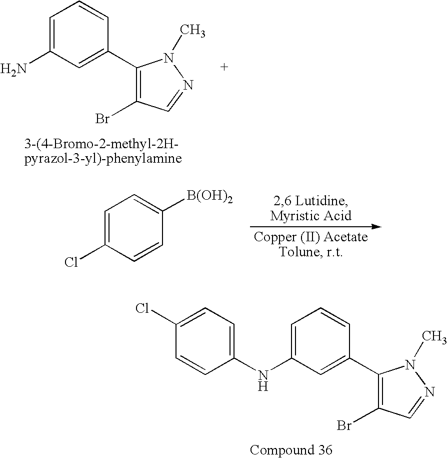

A 3-L three-neck round bottom flask was flushed with argon and filled with ˜200 mL of toluene. Under argon, copper acetate (2.23 g, 12.25 mmol, 0.20 eq.), myristic acid (4.20 g,18.38 mmol, 0.30 eq.), and p-chlorophenylboronic acid (19.1616 g, 122.54 mmol, 2.0 eq.) was added and stirred at room temperature with an oversized stir bar for ten minutes. While mixing, 2,6-lutidine (7.14 mL, 61.27 mmol, 1.0 eq.) was added via syringe and allowed to stir for an additional 10 minutes which enhanced solubility of the reaction mixture. 3-(4-Bromo-2-methyl-2H-pyrazol-3-yl)-phenylamine (15.45 g, 61.27 mmol, 1.0 eq.) was then added and a needle was used to bubble dry air into the reaction mixture overnight. The following evening an additional equivalent of the boronic acid, copper acetate, myristic acid and 2,6-lutidine was added as described above. The reaction mixture was monitored via LC-MS and thin layer chromatography using dichloromethane as the eluent.

The reaction mixture was extracted with a mixture of 4 volumes ethyl acetate, 1.5 volumes of NH₄OH, 0.5 volumes of water and brine. In this mixture an ammonium salt was suspended in the organic layer and as such was filtered off using a Büichner funnel. The ethyl acetate layer was washed twice with NH₄OH and once with distilled water. Sodium sulfate was used to dry the organic layer and the ethyl acetate was removed to yield a crude yellow oil. This product was then purified twice using column chromatography (Biotage 65 M column) with a gradient of 0-5% hexanes in dichloromethane. A third chromatography step was required using a gradient of 10-25% ethyl acetate in hexanes to yield 13.954 g (63% yield) of Compound 36 as an off-white crystalline solid. LCMS m/z (%)=362.0 (MH^{+ 79}Br, 100), 364 (MH^{+ 81}Br, 97). ¹H NMR (400 MHz, DMSO_{d6}): σ8.54 (s, 1H), 7.64 (s, 1H), 7.41 (dd, J=7.9, 1H), 7.29 (dd, J=9.9, 5.3 Hz, 1H), 7.29 (d, J=8.8 Hz, 1H), 7.18-7.10 (m, 3H), 7.13 (d, J=8.9 Hz, 1H) 6.95 (d, J=7.8 Hz, 1H), 3.79 (s, 3H).

### Example 2

### Method B: Synthesis of Compound 54, [3-(4-Bromo-2-methyl-2H-pyrazol-3-yl)-phenyl]-(4-fluoro-2-methyl-phenyl)-amine, using tris(Dibenzylideneacetone)-dipalladium (0) and 2-Bromo-5-Fluorotoluene In the presence of sodium tert-butoxide

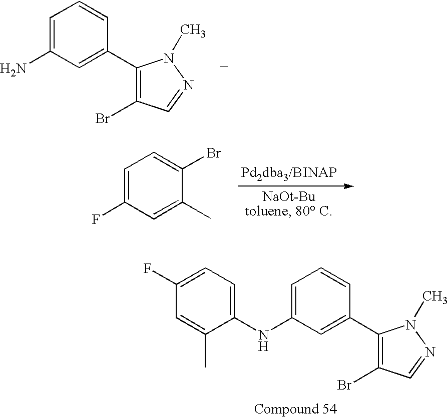

A 20-mL scintillation vial was flushed with argon and then charged of anhydrous toluene (2 mL). Sodium tert-butoxide (53.37 mg, 0.56 mmol), tris(dibenzylideneacetone) dipalladium (18.16 mg, 0.40 mmol), 3-(4-Bromo-2-methyl-2H-pyrazol-3-yl)-phenylamine (0.10 g, 0.40 mmol), 2-bromo-5-fluorotoluene (50.15 μl, 0.40 mmol) and BINAP (26.48 mg, 0.04 mmol). The reaction was heated and stirred at 80° C. for 72 hours under argon atmosphere. The mixture was cooled to ambient temperature, taken up in diethyl ether and ethyl acetate, filtered through celite and concentrated. The crude product was then purified by HPLC. The major peak was collected and one drop of ammonia hydroxide was added to neutralize the acid and lyophilized to produce 74.1 mg (51.4%) of Compound 54 as a tan solid. LCMS m/z (%)=360 (MH^{+ 79}Br, 100), 362 (MH^{+ 81}Br, 97). ¹H NMR (400 MHz, CDCl₃): σ7.70 (s, 1H), 7.33 (dd, J=7.8, 7.8 Hz, 1H), 7.21 (dd, J=8.6, 5.4 Hz, 1H), 6.97 (dd, J=9.2, 2.8 Hz, 1H), 6.91-9.85 (m, 2H), 6.81 (d, J=7.6 Hz, 1H) 6.73 (dd, J=1.8, 1.8 Hz, 1H), 3.84 (s,3H), 2.27 (s, 3H).

### Example 3

### Method C: Synthesis of Compound 61, [3-(4-Bromo-2-methyl-2H-pyrazol-3-yl)-phenyl]-(5-methoxy-benzothiazol-2-yl)-amine, using 2-Chloro-6-methoxy-benzothiazole and Microwave Irradiation (Smith Synthesizer)

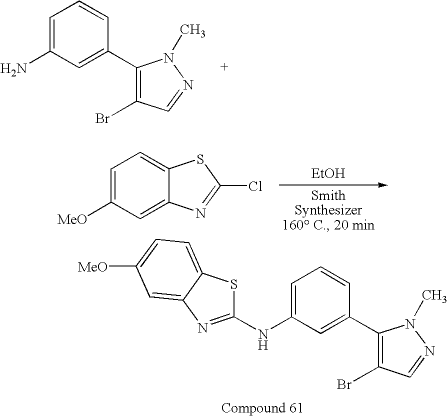

A mixture of 3-(4-bromo-2-methyl-2H-pyrazol-3-yl)-phenylamine (50 mg, 0.2 mmol) and 2-chloro6-methoxy-benzothiazole (39.3 mg, 0.2 mmol) in ethanol (1 mL) was heated on a Smith Synthesizer at 160° C. for 20 minutes. The precipitate that formed was collected by filtration and washed with ethanol to provide 56.8 mg (68.6% yield) of Compound 61 as a white solid: LCMS m/z (%)=415 (MH^{+ 79}Br, 100), 417 (MH^{+ 81}Br, 100).¹H NMR (400 MHz, DMSO-_{d6}): σ10.50 (s, 1H), 7.92 (s, 1H), 7.86 (d, J=8.2 Hz, 1H), 7.68 (s, 1H), 7.54-7.50 (m, 2H), 7.46 (d, J=2.6 Hz, 1H), 7.11 (d, J=7.8 Hz, 1H), 6.93 (dd, J=8.8, 2.6 Hz, 1H), 3.83 (s, 3H), 3.78 (s, 3H).

### Example 4

### Method D: Synthesis of Compound 16, [3-(4Bromo-2-methyl-2H-pyrazol-3-yl)-phenyl]-(2-nitro-phenyl)-amine, using 2-Fluoro-nitrobenzene and heat

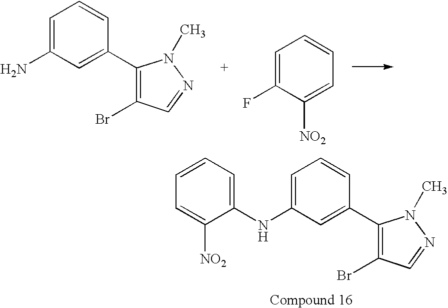

3-(4-Bromo-2-methyl-2H-pyrazol-3-yl)-phenylamine (359 mg, 1.43 mmol, 1 eq) was dissolved in DMA (3 mL) and pyridine (300 μL). To this solution was added 1-fluoro-2-nitrobenzene (180 μL, 1.71 mmol, 1.2 eq) and the mixture was heated to 130° C. for 36 hours. After cooling to ambient temperature, 1N HCl (20 mL) was added and the mixture extracted with Et₂O (3×20 mL). The combined organic layers were dried over Na₂SO₄, filtered and the solvent evaporated. The residue was then purified by SiO₂ Chromatography (Hex→1:1 Hex/EtOAc gradient elution) to provide 24 mg (5%) of Compound 16 as an off white solid: LCMS m/z (%)=373 (MH^{+ 79}Br, 100), 375 (MH^{+ 81}Br, 97). ¹H NMR (400 MHZ, CDCl₃): σ9.55 (s, 1H), 8.22 (d, J=8.4 Hz, 1H), 7.53-7.57 (m, 2H), 7.35-7.42 (m, 3H), 7.23-7.26 (m, 2H), 6.84 (dd, J=7.6, 7.6 Hz, 1H), 3.86 (s,3H).

### Example 5

The following compounds were produced using either Method A, B, C or D or a variation thereof (TABLES 3-4).

TABLE 3

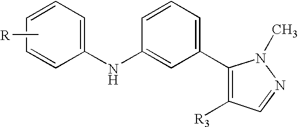

| Cmpd | | | | |
|---|---|---|---|---|
| No. | R | R₃ | Method | LCMS (m/z) |
| 64 | H | Br | A | 328 (MH^{+ 79}Br, 100), 330 |
| | | | | (MH^{+ 81}Br, 100) |
| 17 | 3-Cl | Br | A | 362 (MH^{+ 79}Br, 90), 364 |
| | | | | (MH^{+ 81}Br, 100) |
| 18 | 3,5-diCF₃ | Br | A | 464 (MH^{+ 79}Br, 100), 466 |
| | | | | (MH^{+ 81}Br, 90) |
| 37 | 4-F | Br | A | 346 (MH^{+ 79}Br, 60), 348 |
| | | | | (MH^{+ 81}Br, 100) |
| 38 | 4-OMe | Br | A | 358 (MH^{+ 79}Br, 70), 360 |
| | | | | (MH^{+ 81}Br, 100) |
| 19 | 3-OMe | Br | A | 358 (MH^{+ 79}Br, 70), 360 |
| | | | | (MH^{+ 81}Br 100) |
| 39 | 3,4-methylenedioxy | Br | A | 372 (MH^{+ 79}Br, 100), 374 |
| | | | | (MH^{+ 81}Br, 80) |
| 40 | 3-OCF₃ | Br | A | 412 (MH^{+ 79}Br, 80), 414 |
| | | | | (MH^{+ 81}Br, 100) |
| 41 | 4-Br | Br | A | 406 (MH^{+ 79}Br, 50), 408 |
| | | | | (MH^{+ 81}Br, 100) |
| 20 | 3,4-diOMe | Br | A | 388 (MH^{+ 79}Br, 100), 390 |
| | | | | (MH^{+ 81}Br, 90) |
| 42 | 4-SMe | Br | A | 374 (MH^{+ 79}Br, 80), 376 |
| | | | | (MH^{+ 81}Br, 100) |
| 43 | 4-CN | Br | A | 353 (MH^{+ 79}Br, 100), 355 |
| | | | | (MH^{+ 81}Br, 100) |
| 21 | 3-COCH₃ | Br | A | 370 (MH^{+ 79}Br, 100), 372 |
| | | | | (MH^{+ 81}Br, 100) |
| 22 | 3,5-diCl | Br | A | 396 (MH^{+ 79}Br, 50), 398 |
| | | | | (MH^{+ 81}Br, 100) |
| 23 | 3,5-diMe | Br | A | 356 (MH^{+ 79}Br, 100), 358 |
| | | | | (MH^{+ 81}Br, 90) |
| 44 | 4-CF₃ | Br | A | 396 (MH^{+ 79}Br, 100), |
| | | | | 398 (MH^{+ 81}Br, 100) |
| 45 | 4-OCF₃ | Br | A | 412 (MH^{+ 79}Br, 100), |
| | | | | 414 (MH^{+ 81}Br, 80) |
| 46 | 4-SO₂Me | Br | A | 406 (MH^{+ 79}Br, 100), |
| | | | | 408 (MH^{+ 81}Br, 100) |
| 24 | 3-NHAc | Br | A | 385 (MH^{+ 79}Br, 100), |
| | | | | 387 (MH^{+ 81}Br, 100) |
| 25 | 3-CH₂OH | Br | A | 358 (MH^{+ 79}Br, 90), |
| | | | | 360 (MH^{+ 81}Br, 100) |
| 47 | 3-Cl-4-F | Br | A | 380 (MH^{+ 79}Br, 80), |
| | | | | 382 (MH^{+ 81}Br, 100) |
| 48 | 3,4-diCl | Br | A | 396 (MH^{+ 79}Br, 70), |
| | | | | 398 (MH^{+ 81}Br, 100) |
| 49 | 3-Me-4-Cl | Br | A | 376 (MH^{+ 79}Br, 80), |
| | | | | 378 (MH^{+ 81}Br, 100) |
| 26 | 2-Me-4-Cl | Br | A | 376 (MH^{+ 79}Br, 80), |
| | | | | 378 (MH^{+ 81}Br, 100) |
| 50 | 3,5-diF | Br | A | 364 (MH^{+ 79}Br, 100), |
| | | | | 366 (MH^{+ 81}Br, 100) |
| 51 | 3-Cl-4-CF₃ | Br | A | 430 (MH^{+ 79}Br, 80), |
| | | | | 432 (MH^{+ 81}Br, 100) |
| 52 | 3,4-diF | Br | A | 364 (MH^{+ 79}Br, 100), |
| | | | | 366 (MH^{+ 81}Br, 100) |
| 6 | 4-Cl | Cl | A | 318 (MH^{+ 35}Cl, 100), |
| | | | | 320 (MH^{+ 37}Cl, 60) |
| 7 | 4-CF₃ | Cl | A | 352 (MH^{+ 35}Cl, 100), |
| | | | | 354 (MH^{+ 37}Cl,40) |
| 53 | 3-Me-4-F | Br | A | 360 (MH^{+ 79}Br, 90), |
| | | | | 362 (MH^{+ 81}Br, 100) |
| 27 | 4-OPh | Br | A | 420 (MH^{+ 79}Br, 100), |
| | | | | 422 (MH^{+ 81}Br, 100) |
| 28 | 3-CF₃ | Br | A | 396 (MH^{+ 79}Br, 100), |
| | | | | 398 (MH^{+ 81}Br, 100) |
| 29 | 3-NO₂ | Br | A | 373 (MH^{+ 79}Br, 90), |
| | | | | 375 (MH^{+ 81}Br, 100) |
| 30 | 2,3,4-triOMe | Br | A | 418 (MH^{+ 79}Br, 100), |
| | | | | 420 (MH^{+ 81}Br, 80) |
| 54 | 2-Me-4-F | Br | B | 360 (MH^{+ 79}Br, 90), |
| | | | | 362 (MH^{+ 81}Br, 100) |
| 1 | 4-Cl | H | A | 284 (MH^{+ 35}Cl, 100), |
| | | | | 286 (MH^{+ 37}Cl, 50) |
| 2 | 4-CF₃ | H | A | 318 (MH⁺, 100) |
| 8 | 4-OCF₃ | Cl | A | 368 (MH^{+ 35}Cl, 100), |
| | | | | 370 (MH^{+ 37}Cl, 70) |
| 9 | 3-OCF₃ | Cl | A | 368 (MH^{+ 35}Cl, 100), |
| | | | | 370 (MH^{+ 37}Cl, 70) |
| 10 | 4-F | Cl | A | 302 (MH^{+ 35}Cl, 100), |
| | | | | 304 (MH^{+ 37}Cl, 50) |
| 3 | 4-OCF₃ | H | A | 334 (MH⁺, 100) |
| 4 | 3-OCF₃ | H | A | 334 (MH⁺, 100) |
| 5 | 4-F | H | A | 268 (MH⁺, 100) |
| 31 | 3-F-4-Me | Br | A | 360 (MH^{+ 79}, 90), |
| | | | | 362 (MH^{+ 81}Br, 100) |
| 32 | 2,4-diCF₃ | Br | A | 464 (MH^{+ 79}Br, 100), |
| | | | | 466 (MH^{+ 81}Br, 90) |
| 33 | 3-F-4-OMe | Br | A | 376 (MH^{+ 79}Br, 100), |
| | | | | 378 (MH^{+ 81}Br, 100) |
| 34 | 2,3-diF | Br | A | 364 (MH^{+ 79}Br, 100), |
| | | | | 366 (MH^{+ 81}Br, 100) |
| 35 | 2,4-diF | Br | B | 364 (MH^{+ 79}Br, 100), |
| | | | | 366 (MH^{+ 81}Br, 100) |
| 11 | 3-Me-4-Cl | Cl | A | 332 (MH^{+ 35}Cl, 100), |
| | | | | 334 (MH^{+ 37}Cl, 70) |
| 12 | 3-Cl-4-CF₃ | Cl | A | 386 (MH^{+ 35}Cl, 100), |
| | | | | 388 (MH^{+ 37}Cl, 60) |
| 13 | 3,4-diF | Cl | A | 320 (MH^{+ 35}Cl, 100), |
| | | | | 322 (MH^{+ 37}Cl, 40) |
| 14 | 3-Cl | Cl | A | 318 (MH^{+ 35}Cl, 100), |
| | | | | 320 (MH^{+ 37}Cl, 60) |
| 15 | 4-OMe | Cl | A | 314 (MH^{+ 35}Cl, 100), |
| | | | | 316 (MH^{+ 37}Cl, 40) |
| 65 | 4-I | Br | A | 454 (MH^{+ 79}Br, 100), |
| | | | | 456 (MH^{+ 81}Br, 70) |
| 66 | 2-OMe-5-Me | Br | A | 372 (MH^{+ 79}Br, 80), |
| | | | | 374 (MH^{+ 81}Br, 100) |

TABLE 4

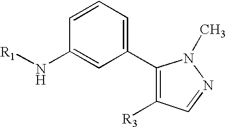

| Cmpd No. | R₁ | R₃ | Method | LCMS (m/z) |
|---|---|---|---|---|
| 55 | 1-naphthyl | Br | A | 378 (MH^{+ 79}Br, 100), 380 |
| | | | | (MH^{+ 81}Br, 100) |
| 56 | 2-naphthyl | Br | A | 378 (MH^{+ 79}Br, 100), 380 |
| | | | | (MH^{+ 81}Br, 100) |
| 63 | 3-quinoline | Br | A | 379 (MH^{+ 79}Br, 100), 381 |
| | | | | (MH^{+ 81}Br, 100) |
| 57 | 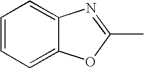 | Br | C | 369 (MH^{+ 79}Br, 100), 371 (MH^{+ 79}Br, 100) |
| 58 | 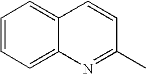 | Br | C | 379 (MH^{+ 79}Br, 100), 381 (MH^{+ 81}Br, 100) |
| 59 | 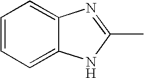 | Br | C | 368 (MH^{+ 79}Br, 100), 370 (MH^{+ 81}Br, 100) |
| 60 | 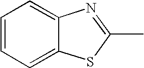 | Br | C | 385 (MH^{+ 79}Br, 90), 387 (MH^{+ 81}Br, 100) |
| 61 | 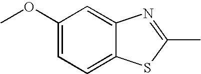 | Br | C | 415 (MH^{+ 79}Br, 100), 417 (MH^{+ 81}Br, 100) |
| 62 | 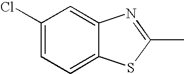 | Br | C | 419 (MH^{+ 79}Br, 70), 421 (MH^{+ 81}Br, 100) |

### Example 6

### Synthesis of Compound 67-[3-(4-Bromo-2-methyl-2H-pyrazol-3-yl)-5-(N,N-dimethylamino)-phenyl]-(4-chloro-phenyl)-amine (Scheme 1)

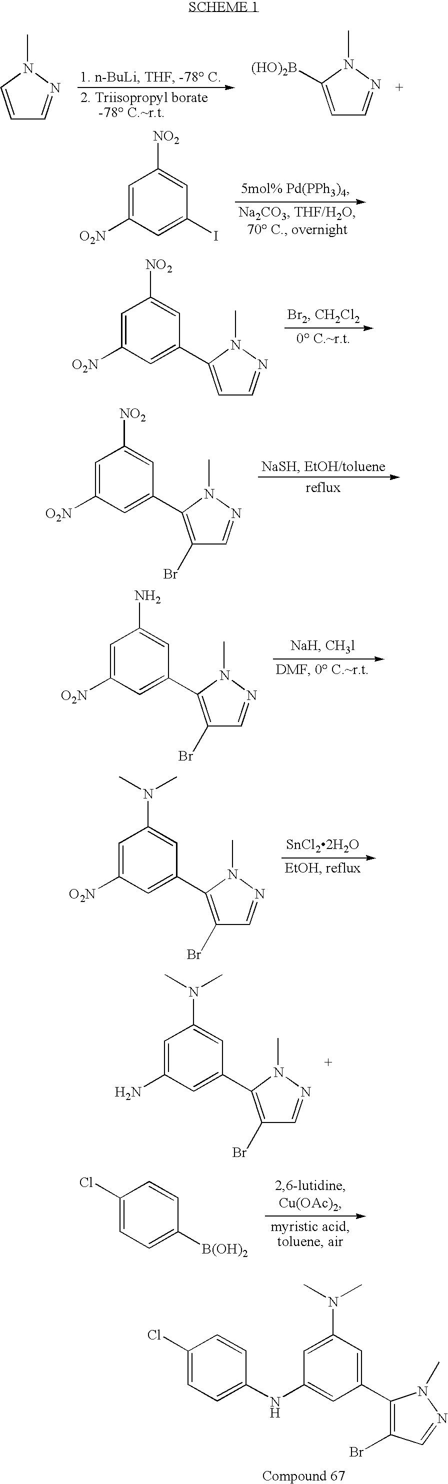

Compound 67 was prepared in an analogous method as described by Method A using 4-chlorophenylboronic acid and 5-(4-bromo-2-methyl-2H-pyrazol-3-yl)-N,N-dimethyl-benzene-1,3-diamine in 25% yield: LCMS m/z (%)=405 (MH^{+ 79}Br, 75), 407 (MH^{+ 81}Br, 100). ¹H NMR (400 MHz, CDCl₃) δ: 7.50 (s, 1H), 7.22 (d, J=8.0 Hz, 2H), 7.04 (d, J=8.0 Hz, 2H), 6.42 (t, J=2.1 Hz, 1H), 6.40 (t, J=1.6 Hz, 1H), 6.29 (dd, J=1.5, 2.3 Hz, 1H), 3.84 (s, 3H), 2.97 (s, 6H).

Intermediate 5-(4-bromo-2-methyl-2H-pyrazol-3-yl)-N,N-dimethyl-benzene-1,3-diamine was prepared by the following steps:

### 1) 2-Methyl-2H-pyrazole-3-boronic acid

To a stirred solution of 1-methylpyrazole (19.80 g, 0.24 mol) in TBF (500 mL) at −78° C. was added n-BuLi (2.5 M in hexane, 116 mL, 0.29 mol, 1.2 eq.) slowly within 30 mins and the mixture was stirred at this temperature for an additional 1.5 hr. Triisopropyl borate (223.0 mL, 181.40 g, 0.96 mmol, 4.0 eq.) was then added dropwise at −78° C., followed by stirring overnight until it was warmed up to r.t. The reaction mixture was acidified to pH=6 with 1N HCl, THF was removed under vacuum and the aqueous residue was extracted with EtOAc (5×400 mL). The combined organic phase was washed with brine, dried over anhydrous MgSO₄, filtered and evaporated. The resultant white solid 2-methyl-2H-pyrazole-3-boronic acid was used without further purification in the subsequent Suzuki cross-coupling step: LCMS m/z (%)=127 (MH⁺, 100). ¹H NMR (400 MHz, CD₃OD) δ: 7.38 (s, 1H), 6.57 (d, J=4.0 Hz, 1H), 3.81 (s, 3H).

### 2) 5-(3,5-Dinitro-phenyl)-1-methyl-1H-pyrazole

A mixture of 1-iodo-3,5-dinitrobenzene (1.200 g, 4.00 mmol), 2-methyl-2H-pyrazole-3-boronic acid (90%, 1.679 g, 10.00 mmol, 3.0 eq.) and Na₂CO₃ (6.783 g, 64.00 mmol, 16.0 eq.) was dissolved in THF (100 mL) and H₂O (75 mL). The mixture was degassed with N₂ for 5 mins. Pd(PPh₃)₄ (0.233 g, 0.20 mmol, 0.05 eq.) was then added, the solution was degassed for an additional 5 mins and the mixture was stirred at 70° C. overnight. After consumption of the stating material, the reaction mixture was diluted with EtOAc (100 mL), and the aqueous phase was extracted with EtOAc three times (3×100 mL). The combined organic phase was washed with brine, dried over anhydrous MgSO₄, filtered and evaporated. The crude reaction mixture was purified by SiO₂ column chromatography (Eluent: EtOAc/Hexane=1/3 then 1/1) providing 5-(3,5-dinitro-phenyl)-1-methyl-1H-pyrazole (0.937 g, 3.78 mmol) in 90% yield: LCMS m/z (%)=249 (MH⁺, 100). ¹H NMR (400 MHz, CDCl₃) δ: 9.08 (t, J=2.0 Hz, 1H), 8.63 (s, 1H), 8.62 (s, 1H), 7.61 (d, J=2.0 Hz, 1H), 6.55 (d, J=1.8 Hz, 1H), 4.01 (s, 3H).

### 3) 4-Bromo-5-(3,5-dinitro-phenyl)-1-methyl-1H-pyrazole

To a stirred solution of 5-(3,5-dinitro-phenyl)-1-methyl-1-H-pyrazole (5.33 g, 21.0 mmol) in CH₂Cl₂ (100 mL) at 0° C. was added dropwise Br₂ (3.77 g, 1.21 mL, 24.0 mmol, 1.1 eq.), the mixture was stirred at 0° C. until the starting material disappeared. The mixture was diluted with EtOAc (200 mL), washed sequentially with saturated NaHCO₃ and saturated Na₂S₂O₃. The aqueous phase was extracted with EtOAc (3×60 mL), the combined organic phase was washed with brine, dried over anhydrous MgSO₄, filtered and evaporated. The crude reaction mixture was triturated with EtOH to provide 4-bromo-5-(3,5-dinitro-phenyl)-1-methyl-1H-pyrazole (4.30 g, 13.0 mmol, 62%) which was directly used in the next selective mono-reduction: LCMS m/z (%)=327 (MH^{+ 79}Br, 80), 329 (MH^{+ 81}Br, 100). ¹H NMR (400 MHz, CDCl₃) δ: 9.17 (t, J=2.0 Hz, 1H), 8.68 (s, 1H), 8.67 (s, 1H), 7.65 (s, 1H), 3.96 (s,3H).

### 4) 3-(4Bromo-2-methyl-2H-pyrazol-3-yl)-5-nitro-phenylamine

To a stirred solution of 4-bromo-5-(3,5-dinitro-phenyl)-1-methyl-1H-pyrazole (4.258 g, 13.0 mmol) in a mixture of methanol/toluene (200 mL/40 mL) at 70° C. was added NaSH in methanol (1.824 g/50 mL, 32.5 mmol, 2.5 eq.) within 45 mins, and the reaction mixture was stirred at this temperature for another 30 mins. The solvent was removed under vacuum, the residue was dissolved in EtOAc, and washed with water and brine. It was dried over anhydrous MgSO₄, filtered and evaporated. The crude reaction mixture was purified by SiO₂ column chromatography (Eluent: EtOAc/Hexane=1/3 then 1/1) to give 3-(4-bromo-2-methyl-2H-pyrazol-3-yl)-5-nitro-phenylamine (3.690 g, 12.0 mmol) in 96% yield: LCMS m/z (%)=297 (MH^{+ 79}Br, 100), 299 (MH^{+ 81}Br, 85). ¹H NMR (400 MHz, CDCl₃) δ: 7.62 (s, 1H), 7.60 (s, 1H), 7.57 (s, 1H), 6.99 (s, 1H), 3.87 (s, 3H).

### 5) [3-(4-Bromo-2-methyl-2H-pyrazol-3-yl)-5-nitro-phenyl]-dimethyl-amine

To a stirred solution of 3-(4-bromo-2-methyl-2H-pyrazol-3-yl)-5-nitro-phenylamine (0.499 g, 1.65 mmol) in DMF (5 mL) at 0° C. was added NaH (60%, 0.264 g, 6.59 mmol, 4.0 eq.) with stirring for 30 mins. CH₃I (0.962 g, 0.42 mL, 6.71 mmol, 4.1 eq.) was added, and the reaction mixture was stirred at this temperature for another 30 mins before warming up to room temperature. The reaction was quenched with EtOH (0.1 mL) and saturated NH₄Cl and extracted with EtOAc (3×20 mL). The combined organic phase was washed with brine, dried over anhydrous MgSO₄, filtered and evaporated. The crude reaction mixture was purified by SiO₂ column chromatography (Eluent: EtOAc/Hexane=1/3) to give [3-(4-bromo-2-methyl-2H-pyrazol-3-yl)-5-nitro-phenyl]-dimethyl-amine (0.181 g, 0.56 mmol, 33%): LCMS m/z (%)=325 (MH^{+ 79}Br, 100), 327 (MH^{+ 81}Br, 85). ¹H NMR (400 MHz, CDCl₃) δ: 7.61 (s, 1H), 7.57.(s, 1H), 6.98 (s, 1H), 3.87 (s, 3), 3.14 (s, 6H).

### 6) 5-(4-Bromo-2-methyl-2H-pyrazol-3-yl)-N,-dimethyl-benzene-1,3-diamine

To a stirred solution of [3-(4-bromo-2-methyl-2H-pyrazol-3-yl)-5-nitro-phenyl]-dimethyl-amine (0.180 g, 0.55 mmol) in EtOH (5 mL) was added SnCl₂.2H₂O (0.509 g, 2.21 mmol, 4.0 eq.) and the mixture was stirred at reflux for 2 hrs. Ethanol was removed under vacuum and the resultant residue was diluted with EtOAc (20 mL), and washed with saturated NaHCO₃. The milky aqueous phase was extracted with EtOAc (3×20 mL), the combined organic phase was dried over anhydrous MgSO₄, filtered and evaporated. After purification by SiO₂ column chromatography (Eluent: EtOAc/Hexane=1/3 then 1/1), 5-(4-bromo-2-methyl-2H-pyrazol-3-yl)-N,N-dimethyl-benzene-1,3-diamine (0.042 g, 0.14 mmol) was obtained in 25% yield: LCMS m/z (%)=295 (MH^{+ 79}Br, 95), 297 (MH^{+ 81}Br, 100). ¹H NMR (400 MHz, CDCl₃) δ: 7.49 (s, 1H), 6.06 (t, J=1.9 Hz, 1H), 6.04 (t J=2.4 Hz, 5.98 (t, J=1.5 Hz, 1H), 3.75 (s, 3H), 2.88 (s, 6H).

### Example 7

### Synthesis of Compound 68-[3-(4-Bromo-2-methyl-2H-pyrazol-3-yl)-5fluoro-phenyl]-(4chloro-phenyl)-amine (Scheme 2)

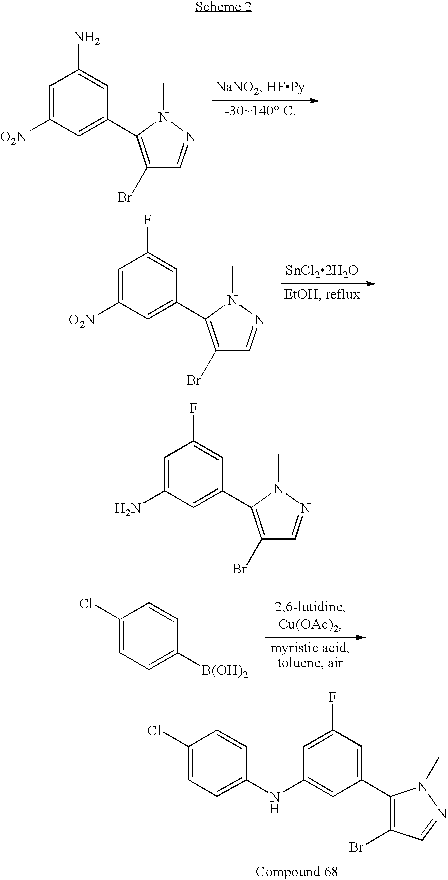

Compound 68 was prepared in an analogous method as described by Method A using 4-chlorophenylboronic acid and 3-(4-bromo-2-methyl-2H-pyrazol-3-yl)-5-fluoro-phenylamine in 67% yield: LCMS m/z (%)=380 (MH^{+ 79}Br, 75), 382 (MH^{+ 81}Br, 100). ¹H NMR (400 MHz, CDCl₃) δ: 7.52 (s, 1H), 7.29 (d, J=8.0 Hz, 2H), 7.08 (d, J=8.0 Hz, 2H), 6.74-6.82 (m, 2H), 6.61 (ddd, J=1.5, 2.2, 8.7 Hz, 1H), 5.98 (broad s, 1H), 3.83 (s, 3H).

Intermediate 3-(4Bromo-2-methyl-2H-pyrazol-3-yl)-5-fluoro-phenylamine was prepared by the following steps:

### 1) 4-Bromo-5(3-fluoro-5-nitro-phenyl)-1-methyl-1H-pyrazole

At 0° C., HF.Py (6 mL) was added to 3-(4-Bromo-2-methyl-2H-pyrazol-3-yl)-5-nitro-phenylamine (1.037 g, 3.49 mmol) dropwise with stirring for 10 mins, then warmed up to r.t. and stirred an additional 30 mins. NaNO₂ (0.265 g, 3.84 mmol, 1.1 eq.) was added at −30° C., and the mixture was stirred at this temperature for 30 mins, it was then heated to 140° C. and stirred for an additional 10 mins. The reaction mixture was cooled to r.t., diluted with EtOAc, washed with water and saturated NaHCO₃ and the aqueous phase was extracted with EtOAc (3×100 mL). The combined organic phase was washed with brine, dried over anhydrous MgSO₄, filtered and evaporated. The crude reaction mixture was purified by SiO₂ column chromatography (Eluent: EtOAc/Hexane=1/5 then 1/3), and 4-bromo-5-(3-fluoro-5-nitro-phenyl)-1-methyl-1H-pyrazole (0.746 g, 2.48 mmol, 71%) was obtained: LCMS m/z (%)=300 (MH^{+ 79}Br, 100), 302 (MH^{+ 81}Br, 98). ¹H NMR (400 MHz, CDCl₃) δ: 8.13 (s, 1H), 8.05 (dt, J=2.1, 7.8 Hz, 1H), 7.58 (s, 1H), 7.52 (ddd, J=1.5, 2.5, 7.1 Hz, 1H), 3.89 (s, 3H).

### 2) 3-(4-Bromo-2-methyl-2H-pyrazol-3-yl)-5-fluoro-phenylamine

To a stirred solution of 4-bromo-5-(3-fluoro-5-nitro-phenyl)-1-methyl-1H-pyrazole (0.524 g, 1.75 mmol) in EtOH (10 mL) was added SnCl₂.2H₂O (1.608 g, 6.98 mmol, 4.0 eq.), the mixture was stirred at reflux for 2 hrs, and EtOH was removed under vacuum. The resultant solid was dissolved in EtOAc, 1N NaOH was added, and the mixture was stirred overnight. The white precipitate was filtered off through celite, and the aqueous phase was extracted with EtOAc (3×50 mL). The combined organic phase was dried over anhydrous MgSO₄, filtered and evaporated. The crude reaction mixture was purified by SiO₂ column chromatography (Eluent: EtOAc/Hexane=1/3) to give 3-(4-bromo-2-methyl-2H-pyrazol-3-yl)-5-fluoro-phenylamine (0.450 g, 1.67 mmol, 95%) as white solid: LCMS m/z (%)=270 (MH^{+ 79}Br, 100), 272 (MH^{+ 81}Br, 80). ¹H NMR (400 MHz, CDCl₃) δ: 7.50 (s, 1H), 6.46 (d, J=1.8 Hz, 1H), 6.44 (s, 2H), 5.84-3.96 (broad s, 21), 3.80 (s, 3H).

### Example 8

### Synthesis of Compound 69-[3-(4-Bromo-2-methyl-2H-pyrazol-3-yl)5-pyrrolidin-1-yl-phenyl]-(4chloro-phenyl)-amine (Scheme 3)

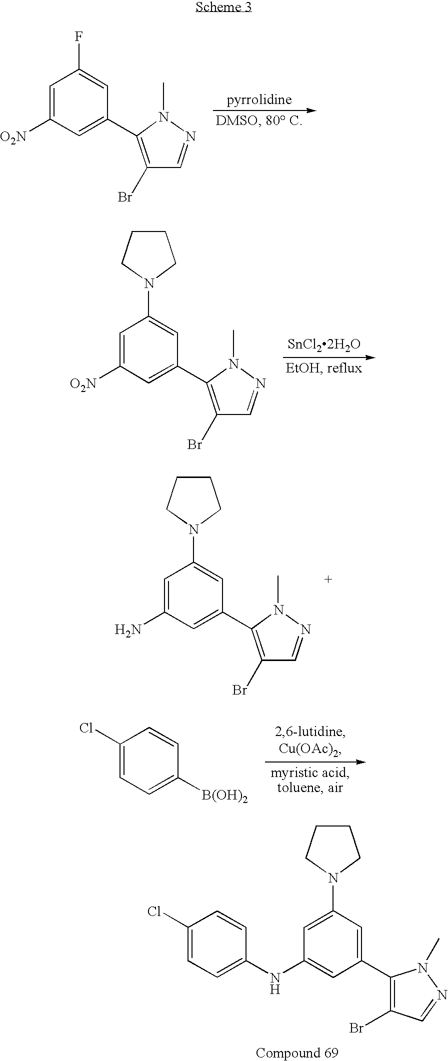

Compound 69 was prepared in an analogous method as described by Method A using 4-chlorophenylboronic acid and 3-(4-bromo-2-methyl-2H-pyrazol-3-yl)-5-pyrrolidin-1-yl-phenylamine in 71% yield by the method A: LCMS mn/z (%)=431 (MH^{+ 79}Br, 50), 433 (MH^{+ 81}Br, 100). ¹H NMR (400 MHz, CDCl₃) δ: 7.50 (s, 1H), 7.21 (d, J=8.0 Hz, 2H), 7.05 (d, J=8.0 Hz, 2H), 6.33 (t, J=1.6 Hz, 1H), 6.27 (t, J=2.3 Hz, 1H), 6.14 (t, J=1.6 Hz, 1H), 5.74 (s, 1H), 3.84 (s, 3H), 3.21 (t, J=6.7 Hz, 4H), 2.01 (dt, J=3.3, 7.0 Hz, 4H).

Intermediate 3-4-bromo-2-methyl-2H-pyrazol-3-yl)-5-pyrrolidin-1-yl-phenylamine was prepared by the following steps:

### 1) 4-Bromo-1-methyl-5-(3-nitro-5-pyrrolidin-1-yl-phenyl)-1H-pyrazole

To a stirred solution of 4-Bromo-5-(3-fluoro-5-nitro-phenyl)-1-methyl-1H-pyrazole (0.217 g, 0.72 mmol) in DMSO (5 mL) was added pyrrolidine (0.25 mL, 0.207 g, 2.89 mmol, 4.0 eq.) and the mixture was stirred at 80° C. for 2 hrs, then cooled down to room temperature and diluted with EtOAc. It was washed with 1N HCl, water and brine, then dried over MgSO₄, filtered and evaporated. The crude reaction mixture was purified by SiO₂ column chromatography (Eluent: EtOAc/Hexane=1/3) to give compound 4-bromo-1-methyl-5-(3-nitro-5-pyrrolidin-1-yl-phenyl)-1H-pyrazole (0.269 g, 0.77 mmol, 89%): LCMS m/z (%)=351 (MH^{+ 79}Br, 100), 353 (MH^{+ 81}Br, 90). ¹H NMR (400 MHz, CDCl₃) δ: 7.55 (d, J=4.0 Hz, 1H), 7.45 (s, 1H), 7.41 (s, 1H), 6.80 (t, J=1.8 Hz, 1H), 3.86 (s, 3H), 3.32 (t, J=6.7 Hz, 4H), 2.02 (dt, J=3.3, 6.6 Hz, 4H).

### 2) 3-(4Bromo-2-methyl-2H-pyrazol-3-yl)-5-pyrrolidin-1-yl-phenylamine

To a stirred solution of 4-bromo-1-methyl-5-(3-nitro-5-pyrrolidin-1-yl-phenyl)-1H-pyrazole (0.249 g, 0.71 mmol) in EtOH (10 mL) was added SnCl₂.2H₂O (0.653 g, 2.84 mmol, 4.0 eq.), the mixture was stirred at reflux for 2 hrs, and EtOH was removed under vacuum. The resultant solid was dissolved in EtOAc, 1N NaOH was added, and the mixture was stirred overnight. The white precipitate was filtered off through celite, and the aqueous phase was extracted with EtOAc (3×50 mL). The combined organic phase was dried over anhydrous MgSO₄, filtered and evaporated. The crude reaction mixture was purified by SiO₂ column chromatography (Eluent: EtOAc/Hexane=1/3 then 1/1) to give 3-(4-bromo-2-methyl-2H-pyrazol-3-yl)-5-pyrrolidin-1-yl-phenylamine (0.208 g, 0.65 mmol, 91%) as white solid: LCMS m/z (%)=321 (MH^{+ 79}Br, 90), 323 (MH^{+ 81}Br, 100). ¹H NMR (400 MHz, CDCl₃) δ: 7.50 (s, 1H), 6.00 (t, J=1.6 Hz, 1H), 5.99 (t, J=1.8 Hz, 1H), 5.97 (t, J=2.0 Hz, 1H), 3.83 (s, 3H), 3.71 (broad s, 2H), 3.28 (t, J=6.7 Hz, 4H), 2.00 (dt, J=3.3, 6.8 Hz, 4H).

### Example 9

### Synthesis of Compound 70-[3-(4-Bromo-2-methyl-2H-pyrazol-3-yl)-4-methoxy-phenyl]-(4-chloro-phenyl)-amine (Scheme 4)

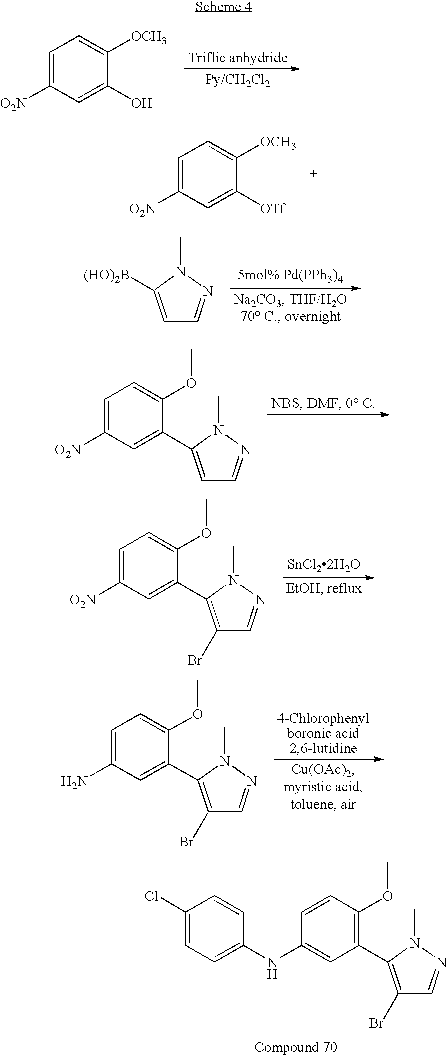

Compound 70 was prepared in an analogous method as described by Method A using 4-chlorophenylboronic acid and 3-(4-bromo-2-methyl-2H-pyrazol-3-yl)-4-methoxy-phenylamine in 41% yield: LCMS m/z (%)=392 (MH^{+ 79}Br, 75), 394 (MH^{+ 81}Br, 100). ¹H NMR (400 MHz, d₆-acetone) δ: 7.48 (s, 1H), 7.45 (s, 1H), 7.28 (dd, J=2.8, 8.8 Hz, 1H), 7.20 (d, J=8.8 Hz, 2H), 7.15 (d, J=8.8 Hz, 1H), 7.08 (d, J=2.8 Hz, 1H), 7.05 (d, J=8.8 Hz, 2H), 3.82 (s, 3H), 3.70 (s, 3H).

Intermediate 3-(4-bromo-2-methyl-2H-pyrazol-3-yl)4-methoxy-phenylamine was prepared by the following steps:

### 1) Trifluoro-methanesulfonic acid 2-methoxy-5-nitro-phenyl ester

To a stirred solution of 2-methoxy-5-nitrophenol (5.092 g, 0.03 mmol) in a mixture of CH₂Cl₂ (3 mL) and pyridine (20 mL) was added triflic anhydride (16.478 g, 9.8 mL, 2.0 eq.) dropwise at 0° C. The mixture was warmed up to r.t. and stirred for 2 hrs. Most of the pyridine was removed under vacuum, the residue was diluted with EtOAc, washed with 1N HCl and water, the aqueous phase was then extracted with EtOAc (3×100 mL). The combined organic phase was washed with brine, dried over anhydrous MgSO₄, filtered and evaporated. The crude reaction mixture was purified by SiO₂ column chromatography (Eluent: EtOAc/Hexane=1/3 then 1/2) to give trifluoro-methanesulfonic acid 2-methoxy-5-nitro-phenyl ester (8.943 g, 0.03 mmol, 100%) as yellow solid: LCMS m/z (%)=302 (MH⁺, 100). ¹H NMR (400 MHz, CDCl₃) δ: 8.30 (dd, J=4.0, 8.0 Hz, 1H), 8.16 (d, J=4.0 Hz, 1H), 7.15 ( d, J=8.0 Hz, 1H), 4.06 (s, 3H).

### 2) 5-(2-Methoxy-5-nitro-phenyl)-1-methyl-1H-pyrazole

Trifluoro-methanesulfonic acid 2-methoxy-5-nitro-phenyl ester (2.561 g, 8.50 mmol), 2-methyl-2H-pyrazole-3-boronic acid (4.283 g,34.01 mmol, 4.0 eq.) and Na₂CO₃ (10.816 g, 102.04 mmol, 12.0 eq.) were dissolved in a mixture of THF (200 mL) and H₂O (100 mL). The mixture was degassed with N₂ for 5 mins, followed by addition of Pd(PPh₃)₄ (0.486 g, 0.42 mmol, 0.05 eq.). After degassing for another 5 mins it was stirred under Ar at 70° C. overnight. Once the reaction was complete, THF was removed and the aqueous phase was extracted with EtOAc (4×100 mL). The combined organic phase was dried over anhydrous MgSO₄, filtered and evaporated. The crude reaction mixture was purified by SiO₂ column chromatography (Eluent: EtOAc/Hexane=1/1) to afford compound 5-(2-methoxy-5-nitro-phenyl)-1-methyl-1H-pyrazole (1.799 g, 7.71 mmol, 91%) as white solid: LCMS m/z (%)=234 (MH⁺, 100). ¹H NMR (400 MHz, CDCl₃) δ: 8.34 (dd, J=2.8, 9.2 Hz, 1H), 8.19 (d, J=2.8 Hz, 1H), 7.56 (d, J=2.0 Hz, 1H), 7.08 (d, J=9.2 Hz, 1H), 6.31 (d, J=1.6 Hz, 1H), 3.96 (s, 3H), 3.74 (x, 3H).

### 3) 4-Bromo-5-(2-methoxy-5-nitro-phenyl)-1-methyl-1H-pyrazole

To a stirred solution of 5-(2-methoxy-5-nitro-phenyl)-1-methyl-1H-pyrazole (1.787 g, 7.66 mmol) in DMF (20 mL) was added NBS (1.515 g, 8.43 mmol, 1.1 eq.) in DMF (5 mL) dropwise at 0° C. Stirring at 0° C. for 3 hrs and TLC showed the completion of the reaction. It was diluted with EtOAc (300 mL), washed with water (3×10 mL) and brine. The EtOAc phase was dried over anhydrous MgSO₄, filtered and evaporated. The crude reaction mixture was purified by SiO₂ column chromatography (Eluent: EtOAc/Hexane=1/3 then 1/1) to give the product 4-bromo-5-(2-methoxy-5-nitro-phenyl)-1-methyl-1H-pyrazole (2.214 g, 7.09 mmol, 93%) as light yellow solid: LCMS m/z (%)=312 (MH^{+ 79}Br, 100), 314 (MH^{+ 81}Br, 100). ¹H NMR (400 MHz, CDCl₃) δ: 8.40 (dd, J=2.4, 6.9 Hz, 1H), 8.22 (m, 1H), 7.57 (s, 1H), 7.14 (d, J=9.2 Hz, 1H), 3.98 (s, 3H), 3.74 (s, 3H).

### 4) 3-(4-Bromo-2-methyl-2H-pyrazol-3-yl)-4-methoxy-phenylamine

To a stirred solution of 4-bromo-5-(2-methoxy-5-nitro-phenyl)-1-methyl-1H-pyrazole (1.799 g, 5.76 mmol) in EtOH (20 mL) was added SnCl₂.2H₂O (5.306 g, 23.05 mmol, 4.0 eq.), the mixture was stirred at reflux for 2 hrs, and EtOH was removed under vacuum. The resultant solid was dissolved in EtOAc, 1N NaOH (30 mL) was added, and the mixture was stirred overnight. The white precipitate was filtered off through celite, and the aqueous phase was extracted with EtOAc (3×80 mL). The combined organic phase was dried over anhydrous MgSO₄, filtered and evaporated. The crude reaction mixture was purified by SiO₂ column chromatography (Eluent: EtOAc/Hexane=1/3 then 1/1) to give 3-4-bromo-2-methyl-2H-pyrazol-3-yl)-4-methoxy-phenylamine (1.430 g, 5.07 mmol, 88%) as white solid: LCMS m/z (%)=282 (MH^{+ 79}Br, 98), 284 (MH^{+ 81}Br, 100). ¹H NMR (400 MHz, CDCl₃) δ: 7.52 (s, 1H), 6.86 (d, J=8.8 Hz, 1H), 6.80 (dd, J=2.8, 8.8 Hz, 1H), 6.22 (d, J=2.4 Hz, 1H), 4.25 (broad s, 2H), 3.72 (s, 3H), 3.71 (s, 3H).

### EXAMPLE 10

### Intracellular IP₃ Accumulation Assay

It is well established that human 5-HT_{2A} receptors coupled to the guanine nucleotide binding protein Gq. 5-HT_{2A}-mediated activation of Gq results in activation of membrane associated phospholipase C which in turn catalyzes the hydrolysis of phosphatidylinositol species (PI, PIP, and PIP2) to form inositol phosphate species (IP, IP2, and IP3). The accumulation of inositol phosphate species can be measured and is often used to determine the functional potency of 5-HT_{2A} agonists or antagonists. However, to evaluate compounds for potential inverse agonist activity, the 5-HT_{2A} receptor must display constitutive activity. We have mutated the human 5-HT_{2A} receptor so as to generate a non-endogenous, constitutively activated version of the 5-HT_{2A} receptor, as disclosed in SEQ ID NO:30 (polynucleotide) and SEQ ID NO:31 (polypeptide) in U.S. Pat. No. 6,541,209 (the disclosure of which is hereby incorporated by reference in its entirety). Upon transfection of this mutated 5-HT_{2A} into HEK293 cells, it was observed that there was a ligand-independent increase in basal inositol phosphate accumulation compared to untransfected cells. This ligand-independent insoitol phosphate accumulation is a trademark of constitutive activity. The following protocol was used to assess inverse agonist potency of compounds.

On day one, 13×10⁶ HEK293 cells per 150 mm plate were plated out. On day two, 2 ml of serum OptimemI is added per plate followed by addition of 60 ul of lipofectamine and 16ug of cDNA. Note that lipofectamine must be added to the OptimemI and mixed well before addition of cDNA. While complexes between lipofectamine and the cDNA are forming, media is carefully aspirated and cells are gently rinsed with 5 ml of OptimemI media followed by careful aspiration. Then 12 ml of OptimemI is added to each plate and 2 ml of transfection solution is added followed by a 5 hour incubation at 37° C. in a 5% CO₂ incubator. Plates are then carefully aspirated and 25 ml of Complete Media are added to each plate and cells are then incubated until used. On day 3, cells are trypsinized with 5 ml of 0.05% trypsin for 20-30 seconds followed by addition of 10 ml of warmed media, gently titurated to dissociate cells, and then 13 additional ml of warmed media is gently added. Cells are then counted and then 55,000 cells are added to 96-well sterile poly-D-lysine coated plates. Cells are allowed to attach over a six hour incubation at 37° C. in a 5% CO₂ incubator. Media is then carefuilly aspirated and 100 ul of warm inositol-free media plus 0.5 μCi ³H-inositol is added to each well and the plates are incubated for 18-20 hours at 37° C. in a 5% CO₂ incubator.

On day 4, media is carefully aspirated and then 0.1 ml of assay medium is added containing inositol-free/serum free media, 10 μM pargyline, 10 mM lithium chloride, and test compound at indicated concentrations. The plates were then incubated for three hours at 37° C. and then wells are carefully aspirated. Then 200 ul of ice-cold 0.1M formic acid is added to each well. Plates can then be frozen at this point at −80° C. until further processed. Frozen plates are then thawed over the course of one hour, and the contents of the wells (approximately 220 ul) are placed over 400 ul of washed ion-exchange resin (AG 1-X8) contained in a Multi Screen Filtration plate and incubated for 10 minutes followed by filtration under vacuum pressure. Resin is then washed nine times with 200 ul of water and then [³H]inositol phosphates are eluted into a collecting plate by the addition of 200 ul of 1M ammonium formate and an additional 5 minute incubation. The elutant is then transferred to 20 ml scintillation vials, 8 ml of SuperMix or Hi-Safe scintillation cocktails is added, and vials are counted for 0.5-1 minutes in a Wallac 1414 scintilation counter.

Inverse agonist IC₅₀ values (the concentration of test compound that inhibits the constitutive inositol phosphate accumulation by 50%) were determined in the human CART 5-HT2A inositol phosphate assay by testing the test compound at 7-8 different concentrations typically ranging from 0.01 nM to 10 uM. At each concentration, triplicate determinations were made. The mean value of inositol phosphate accumulation at each test compound concentration is calculated and then the data are fit to a non-linear curve-fitting program that allows calculation of the IC₅₀ value.

### Example 11

### Activity for the Compounds of the Present Invention in the IP Accumulation Assay

Certain compounds of the present invention and their corresponding activities in the IP Accumulation Assay are shown in TABLE 5.

TABLE 5

| | 5-HT_{2A} (IC₅₀)* |
|---|---|
| Compound No. | IP Accumulation Assay (nM) |
| 8 | 44.6 |
| 36 | 7.00 |
| 44 | 2.99 |
| 46 | 113 |
| 49 | 19.0 |
| 57 | 10.55 |
| 70 | 1.77 |

*Reported values are averages of at least two trials.

The majority of the other compounds of the Examples were tested at least once, and they showed activities in the 5-HT_{2A} IP Accumulation Assay in the range between about 4 nM and about 10 μM.

1-97. (canceled)

98. A compound of Formula (A): 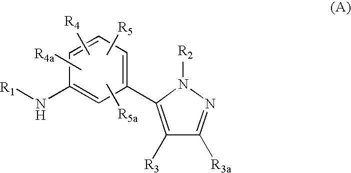
wherein:
i) R₁ is aryl or heteroaryl each optionally substituted with 1 to 5 substituents selected independently from the group consisting of C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, amino, C₁₋₄ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol, nitro, phenoxy and phenyl; and wherein C₂₋₆ alkenyl, C₁₋₆ alkyl and C₂₋₆ alkynyl substituents may be optionally substituted with 1 to 5 substituents selected independently from the group consisting of C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, amino, C₁₋₄ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro; or two adjacent substituents together with the ring carbons to which they are bonded form a C₅₋₇ cycloalkyl optionally replaced with 1 to 2 oxygen atoms;
ii) R₂ is C₁₋₆ alkyl, C₂₋₆ alkenyl, C₂₋₆ alkynyl or C₃₋₇ cycloalkyl;
iii) R₃ is H, C₂₋₆ alkenyl, C₁₋₆ alkyl, C₁₋₅ alkylcarboxamide, C₂₋₆ alkynyl, C₁₋₄ alkylsulfonamide, carbo-C₁₋₆-alkoxy, carboxamide, carboxy, cyano, C₃₋₇ cycloalkyl, C₂₋₈ dialkylcarboxamide, halogen, heteroaryl or phenyl; and wherein C₂₋₆ alkenyl, C₁₋₆ alkyl, C₂₋₆ alkynyl, C₁₋₄ alkylsulfonamide, C₃₋₇ cycloalkyl, heteroaryl or phenyl may be optionally substituted with 1 to 5 substituents selected independently from the group consisting of C₂₋₆ alkenyl, C₁₋₆ alkyl, C₁₋₄ alkoxy, amino, C₁₋₄ alkylamino, C₂₋₆ alkynyl, C₂₋₈ dialkylamino, halogen, C₁₋₄ haloalkoxy, C₁₋₄ haloalkyl, hydroxyl and thiol;
iv) R₃ₐ is selected from the group consisting of H, C₁₋₆ acyl, C₁₋₆ acyloxy, C₂₋₆ alkenyl, C₁₋₆ alkoxy, C₁₋₆ alkyl, C₁₋₆ alkylcarboxamide, C₂₋₆ alkynyl, C₁₋₆ alkylsulfonamide, C₁₋₆ alkylsulfinyl, C₁₋₆ alkylsulfonyl, C₁₋₆ alkylthio, C₁₋₆ alkylureyl, amino, C₁₋₆ alkylamino, C₂₋₈ dialkylamino, carbo-C₁₋₆-alkoxy, carboxamide, carboxy, cyano, C₃₋₇ cycloalkyl, C₂₋₈ dialkylcarboxamide, C₂₋₈ dialkylsulfonamide, halogen, C₁₋₆ haloalkoxy, C₁₋₆ haloalkyl, C₁₋₆ haloalkylsulfinyl, C₁₋₆ haloalkylsulfonyl, C₁₋₆ haloalkylthio, hydroxyl, thiol, nitro and sulfonamide; and
V) R₄, R₄ₐ, R₅ and R₅ₐ are each independently H, C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol, 5 or 6 membered-heteroaryl, nitro, phenyl or NR₆R₇, and where the 5 or 6 membered-heteroaryl or phenyl is optionally substituted with a substituents selected from the group consisting of H, C₁₋₅ acyl, C₁₋₅ acyloxy, C₂₋₆ alkenyl, C₁₋₄ alkoxy, C₁₋₆ alkyl, C₁₋₅ alkylcarboxamide, C₂₋₆ alkynyl, C₁₋₄ alkylsulfonamide, C₁₋₄ alkylsulfinyl, C₁₋₄ alkylsulfonyl, C₁₋₄ alkylthio, C₁₋₆ alkylureyl, carbo-C₁₋₆-alkoxy, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro;
wherein:
R₆ and R₇ are each independently selected from the group consisting of H, C₁₋₆ alkyl, C₂₋₆ alkenyl, C₃₋₇ cycloalkyl, phenyl and benzyl group; wherein each said C₁₋₆ alkyl, C₂₋₆ alkenyl, C₃₋₇ cycloalkyl, phenyl and benzyl group is optionally substituted with 1 to 5 substituents selected independently from the group consisting of H, C₁₋₅ acyl, C₂₋₆ alkenyl, C₁₋₄ alkoxy, C₁₋₆ alkyl, C₁₋₅ alkylcarboxamide, C₁₋₄ alkylthio, carbo-C₁₋₆-alkoxy, amino, C₁₋₄ alkylamino, C₂₋₈ dialkylamino, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro; or
R₆ and R₇ together with the nitrogen to which they are bonded form a 5, 6 or 7 membered cyclic structure which can be saturated or unsaturated and can contain up to four heteroatoms selected from O, NR₈ or S and said cyclic structure may be optionally substituted with 1 to 5 substituents selected independently from the group consisting of H, C₁₋₅ acyl, C₂₋₆ alkenyl, C₁₋₄ alkoxy, C₁₋₆ alkyl, C₁₋₅ alkylcarboxamide, C₁₋₄ alkylthio, carbo-C₁₋₆-alkoxy, amino, C₁₋₄ alkylamino, C₂₋₈ dialkylamino, carboxamide, carboxy, cyano, C₃₋₇ cycloalkyl, C₂₋₈ dialkylcarboxamide, halogen, C₁₋₄ haloalkoxy, C₁₋₄ haloalkyl, C₁₋₄ haloalkylsulfinyl, C₁₋₄ haloalkylsulfonyl, C₁₋₄ haloalkylthio, hydroxyl, thiol and nitro;
R₈ is H or C₁₋₆ alkyl; or
a pharmaceutically acceptable salt, hydrate or solvate thereof.

99. The compound according to claim 98, having Formula (I):

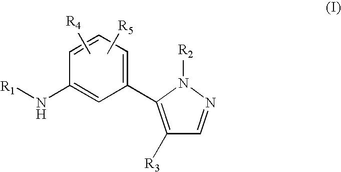

100. The compound according to claim 98, wherein R₁ is aryl optionally substituted with 1 to 5 substituents selected independently from the group consisting of C₁₋₅ acyl, C₁₋₄ alkoxy, C₁₋₆ alkyl, C₁₋₅ alkylcarboxamide, C₁₋₄ alkylsulfinyl, C₁₋₄ alkylsulfonyl, C₁₋₄ alkylthio, amino, C₁₋₄ alkylamino, C₂₋₈ dialkylamino, carboxamide, carboxy, carbo-C₁₋₆-alkoxy, cyano, C₂₋₈ dialkylcarboxamide, halogen, C₁₋₄ haloalkoxy, C₁₋₄ haloalkyl, hydroxyl, thiol, nitro and phenoxy; and where C₁₋₆ alkyl is optionally substituted with 1 to 3 substituents selected from the group consisting of C₁₋₄ alkoxy, C₁₋₅ alkylcarboxamide, amino, C₁₋₄ alkylamino, C₂₋₈ dialkylamino, halogen, C₁₋₄ haloalkoxy, C₁₋₄ haloalkyl, hydroxyl and thiol.

101. The compound according to claim 98, wherein R₁ is aryl optionally substituted with 1 to 5 substituents selected independently from the group consisting of NO₂, F, Cl, Br, I, CF₃, CF₂CF₃, OCH₃, OCH₂CH₃, OCF₃, OCF₂CF₃, SCH₃, SCH₂CH₃, S(O)CH₃, S(O)CH₂CH₃, S(O)₂CH₃, S(O)₂CH₂CH₃, CO₂H, CN, COCH₃, COCH₂CH₃, CH₃, CH₂CH₃, NHCOCH₃, CH₂OH and OC₆H₅.

102. The compound according to claim 98, wherein R₁ is phenyl optionally substituted with 1 to 5 substituents selected independently from the group consisting of NO₂, F, Cl, Br, I, CF₃, CF₂CF₃, OCH₃, OCH₂CH₃, OCF₃, OCF₂CF₃, SCH₃, SCH₂CH₃, S(O)CH₃, S(O)CH₂CH₃, S(O)₂CH₃, S(O)₂CH₂CH₃, CO₂H, CN, COCH₃, COCH₂CH₃, CH₃, CH₂CH₃, NHCOCH₃, CH₂OH and OC₆H₅.

103. The compound according to claim 98, wherein R₁ is selected from the group consisting of benzoxazolyl-2-yl, quinolin-2-yl, quinolin-3-yl, benzimidazol-2-yl, and benzothiazol-2-yl each optionally substituted with 1 to 3 substituents selected independently from the group consisting of C₁₋₄ alkoxy, C₁₋₆ alkyl, amino, C₁₋₄ alkylamino, C₂₋₈ dialkylamino, carbo-C₁₋₆-alkoxy, carboxamide, carboxy, halogen, C₁₋₄ haloalkoxy, C₁₋₄ haloalkyl, hydroxyl, thiol and nitro.

104. The compound according to claim 103, wherein R₁ is selected from the group consisting of benzoxazol-2-yl, quinolin-2-yl, benzoimidazol-2-yl, benzothiazol-2-yl, 5-methoxy-benzothiazol-2-yl, 5-chloro-benzothiazol-2-yl, and quinolin-3-yl.

105. The compound according to claim 98, wherein R₂ is C₁₋₆ alkyl.

106. The compound according to claim 105 wherein R₂ is CH₃.

107. The compound according to claim 98 wherein R₃ is H, Cl, Br, CO₂CH₃, CO₂CH₂CH₃, 2-hydroxyethyl, 2-dimethylaminoethyl, 2-diethylaminoethyl, vinyl, CH₃, CH₂CH₃, phenyl, 4-methoxyphenyl, 3-methoxyphenyl, 4-fluorophenyl, 4-trifluoromthoxyphenyl, thiophenyl, CO₂H, cyclopropyl, —CCH, —CH═CH—CCH or CN.

108. The compound according to claim 107 wherein R₃ is H, Cl or Br.

109. The compound according to claim 98 wherein R₄ is H, halogen or NR₆R₇.

110. The compound according to claim 109 wherein R₄ is H, F, N(CH₃)₂, or pyrrolidin-1-yl.

111. The compound according to claim 98 wherein R₅ is H.

112. The compound according to claim 98, having Formula (II): 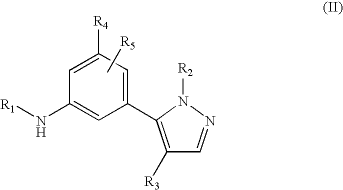
wherein:
R₄ is H, C₁₋₄ alkoxy, phenyl, halogen, 5 or 6 membered-heteroaryl, hydroxyl, thiol or NR₆R₇, where the phenyl or heteroaryl group is optionally substituted with 1 to 5 substituents independently selected from the group consisting of C₁₋₅ acyl, C₁₋₄ alkoxy, C₁₋₆ alkyl, C₁₋₅ alkylcarboxamide, C₁₋₄ alkylsulfonyl, C₁₋₄ alkylthio, amino, C₁₋₄ alkylamino, C₂₋₈ dialkylamino, cyano, C₂₋₈ dialkylcarboxamide, halogen, C₁₋₄ haloalkoxy, C₁₋₄ haloalkyl, hydroxyl, thiol and nitro; and
wherein:
R₆ and R₇ are independently H, C₁₋₆ alkyl, or
R₆ and R₇ together with the nitrogen to which they are bonded form a 5, 6 or 7 membered cyclic structure that may contain up to four heteroatoms selected from O, S or N—C₁₋₄ alkyl; and
R₅ is H, C₁₋₄ alkoxy, C₁₋₆ alkyl, carboxamide, carboxy, cyano, halogen, C₁₋₄ haloalkoxy, C₁₋₄ haloalkyl, hydroxyl, thiol or nitro.

113. The compound according to claim 112, wherein R₄ is H, Cl, F, dimethylamino, diethylamino, pyrrolidin-1-yl, morpholin-1-yl, 4-methylpiperazin-1-yl, 4-ethylpiperazin-1-yl, hydroxyl, thiol, OCH₃ or OCH₂CH₃; and R₅ is H or halogen.

114. The compound according to claim 98, having Formula (IVa): 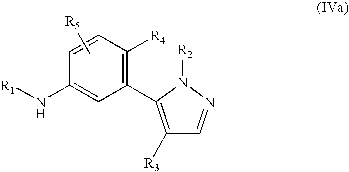
wherein:
R₄ is H, or C₁₋₄ alkoxy; and
R₅ is H, C₁₋₄ alkoxy, C₁₋₆ alkyl, carboxamide, carboxy, cyano, halogen, C₁₋₄ haloalkoxy, C₁₋₄ haloalkyl, hydroxyl, thiol or nitro.

115. The compound according to claim 114, wherein R₄ is OCH₃, and R₅ is H.

116. The compound according to claim 98, selected from the group consisting of:
(4-Chloro-phenyl)-[3-(2-methyl-2H-pyrazol-3-yl)-phenyl]-amine;
[3-(2-Methyl-2H-pyrazol-3-yl)-phenyl]-(4-trifluoromethyl-phenyl)-amine;
[3-(2-Methyl-2H-pyrazol-3-yl)-phenyl]-(4-trifluoromethoxy-phenyl)-amine;
[3-(2-Methyl-2H-pyrazol-3-yl)-phenyl]-(3-trifluoromethoxy-phenyl)-amine;
[3-(2-Methyl-2H-pyrazol-3-yl)-phenyl]-(4-fluoro-phenyl)-amine;
(4-Chloro-phenyl)-[4-methoxy-3-(2-methyl-2H-pyrazol-3-yl)-phenyl]-amine;
(4-Chloro-phenyl)-[3-(2-isopropyl-2H-pyrazol-3-yl)-phenyl]-amine;
(4-Fluoro-phenyl)-[3-(2-isopropyl-2H-pyrazol-3-yl)-phenyl]-amine;
[3-(4-Chloro-2-methyl-2H-pyrazol-3-yl)-phenyl]-(4-chloro-phenyl)-amine;
[3-(4-Chloro-2-methyl-2H-pyrazol-3-yl)-phenyl]-(4-trifluoromethyl-phenyl)-amine;
[3-(4-Chloro-2-methyl-2H-pyrazol-3-yl)-phenyl]-(4-trifluoromethoxy-phenyl)-amine;
[3-(4-Chloro-2-methyl-2H-pyrazol-3-yl)-phenyl]-(3-trifluoromethoxy-phenyl)-amine;
[3-(4-Chloro-2-methyl-2H-pyrazol-3-yl)-phenyl]-(4-fluoro-phenyl)-amine;
[3-(4-Chloro-2-methyl-2H-pyrazol-3-yl)-phenyl]-(3-methyl-4-chloro-phenyl)-amine;
[3-(4-Chloro-2-methyl-2H-pyrazol-3-yl)-phenyl]-(3-chloro-4-trifluoromethyl-phenyl)-amine;
[3-(4-Chloro-2-methyl-2H-pyrazol-3-yl)-phenyl]-(3,4-difluoro-phenyl)-amine;
[3-(4-Chloro-2-methyl-2H-pyrazol-3-yl)-phenyl]-(3-chloro-phenyl)-amine;
[3-(4-Chloro-2-methyl-2H-pyrazol-3-yl)-phenyl]-(4-methoxy-phenyl)-amine;
[3-(4-Chloro-2-methyl-2H-pyrazol-3-yl)4-methoxy-phenyl]-(4-chloro-phenyl)-amine;
(4-Chloro-phenyl)-[3-(4-fluoro-2-methyl-2H-pyrazol-3-yl)4-methoxy-phenyl]-amine;
(4-Chloro-phenyl)-[3-(4-fluoro-2-methyl-2H-pyrazol-3-yl)-phenyl]-amine;
[3-(4-Bromo-2-methyl-2H-pyrazol-3-yl)-phenyl]-(2-nitro-phenyl)-amine;
[3-(4-Bromo-2-methyl-2H-pyrazol-3-yl)-phenyl]-(3-chloro-phenyl)-amine;
[3-(4-Bromo-2-methyl-2H-pyrazol-3-yl)-phenyl]-(3,5-bis-trifluoromethyl-phenyl)-amine;
[3-(4-Bromo-2-methyl-2H-pyrazol-3-yl)-phenyl]-(3-methoxy-phenyl)-amine;
[3-(4-Bromo-2-methyl-2H-pyrazol-3-yl)-phenyl]-(3,4-dimethoxy-phenyl)-amine;
1-{3-[3-(4-Bromo-2-methyl-2H-pyrazol-3-yl)-phenylamino]-phenyl}-ethanone;
[3-(4-Bromo-2-methyl-2H-pyrazol-3-yl)-phenyl]-(3,5-dichloro-phenyl)-amine;
[3-(4-Bromo-2-methyl-2H-pyrazol-3-yl)-phenyl]-(3,5-dimethyl-phenyl)-amine;
N-{3-[3-(4-Bromo-2-methyl-2H-pyrazol-3-yl)-phenylamino]-phenyl}-acetamide;
{3-[3-(4-Bromo-2-methyl-2H-pyrazol-3-yl)-phenylamino]-phenyl }-methanol;
[3-(4-Bromo-2-methyl-2H-pyrazol-3-yl)-phenyl]-(2-methyl-4-chloro-phenyl)-amine;
[3-(4-Bromo-2-methyl-2H-pyrazol-3-yl)-phenyl]-(4-phenoxy-phenyl)-amine;
[3-(4-Bromo-2-methyl-2H-pyrazol-3-yl)-phenyl]-(3-trifluoromethyl-phenyl)-amine;
[3-(4-Bromo-2-methyl-2H-pyrazol-3-yl)-phenyl]-(3-nitro-phenyl)-amine;
[3-(4-Bromo-2-methyl-2H-pyrazol-3-yl)-phenyl]-(2,3,4-trimethoxy-phenyl)-amine;
[3-(4-Bromo-2-methyl-2H-pyrazol-3-yl)-phenyl]-(3-fluoro-4-methyl-phenyl)-amine;
[3-(4-Bromo-2-methyl-2H-pyrazol-3-yl)-phenyl]-(2,4-bis-trifluoromethyl-phenyl)-amine;
[3-(4-Bromo-2-methyl-2H-pyrazol-3-yl)-phenyl]-(3-fluoro-4-methoxy-phenyl)-amine;
[3-(4-Bromo-2-methyl-2H-pyrazol-3-yl)-phenyl]-(2,3-difluoro-phenyl)-amine;
[3-(4-Bromo-2-methyl-2H-pyrazol-3-yl)-phenyl]-(2,4-difluoro-phenyl)-amine;
[3-(4-Bromo-2-methyl-2H-pyrazol-3-yl)-phenyl]-(4-chloro-phenyl)-amine;
[3-(4-Bromo-2-methyl-2H-pyrazol-3-yl)-phenyl]-(4-fluoro-phenyl)-amine;
[3-(4-Bromo-2-methyl-2H-pyrazol-3-yl)-phenyl]-(4-methoxy-phenyl)-amine;
Benzo[1,3]dioxol-5-yl-[3-(4-bromo-2-methyl-2H-pyrazol-3-yl)-phenyl]-amine;
[3-(4-Bromo-2-methyl-2H-pyrazol-3-yl)-phenyl]-(3-trifluoromethoxy-phenyl)-amine;
[3-(4-Bromo-2-methyl-2H-pyrazol-3-yl)-phenyl]-(4-bromo-phenyl)-amine;
[3-(4-Bromo-2-methyl-2H-pyrazol-3-yl)-phenyl]-(4-methylsulfanyl-phenyl)-amine;
4-[3-(4-Bromo-2-methyl-2H-pyrazol-3-yl)-phenylamino]-benzonitrile;
[3-(4-Bromo-2-methyl-2H-pyrazol-3-yl)-phenyl]-(4-trifluoromethyl-phenyl)-amine;
[3-(4-Bromo-2-methyl-2H-pyrazol-3-yl)-phenyl]-(4-trifluoromethoxy-phenyl)-amine;
[3-(4-Bromo-2-methyl-2H-pyrazol-3-yl)-phenyl]-(4-methanesulfonyl-phenyl)-amine;
[3-(4-Bromo-2-methyl-2H-pyrazol-3-yl)-phenyl]-(3-chloro-4-fluoro-phenyl)-amine;
[3-(4-Bromo-2-methyl-2H-pyrazol-3-yl)-phenyl]-(3,4-dichloro-phenyl)-amine;
[3-(4-Bromo-2-methyl-2H-pyrazol-3-yl)-phenyl]-(3-methyl4-chloro-phenyl)-amine;
[3-(4-Bromo-2-methyl-2H-pyrazol-3-yl)-phenyl]-(3,5-difluoro-phenyl)-amine;
[3-(4-Bromo-2-methyl-2H-pyrazol-3-yl)-phenyl]-(3-chloro-4-trifluoromethyl-phenyl)-amine;
[3-(4-Bromo-2-methyl-2H-pyrazol-3-yl)-phenyl]-(3,4-difluoro-phenyl)-amine;
[3-(4-Bromo-2-methyl-2H-pyrazol-3-yl)-phenyl]-(3-methyl-4-fluoro-phenyl)-amine;
[3-(4-Bromo-2-methyl-2H-pyrazol-3-yl)-phenyl]-(2-methyl-4-fluoro-phenyl)-amine;
[3-(4-Bromo-2-methyl-2H-pyrazol-3-yl)-phenyl]-(4-iodo-phenyl)-amine;
[3-(4-Bromo-2-methyl-2H-pyrazol-3-yl)-phenyl]-(2-methoxy-5-methyl-phenyl)-amine;
[3-(4-Bromo-2-methyl-2H-pyrazol-3-yl)-5-(N,N-dimethylamino)-phenyl]-(4-chloro-phenyl)-amine;
[3-(4-Bromo-2-methyl-2H-pyrazol-3-yl)-5-fluoro-phenyl]-(4-chloro-phenyl)-amine;
[3-(4-Bromo-2-methyl-2H-pyrazol-3-yl)-5-pyrrolidin-1-yl-phenyl]-(4-chloro-phenyl)-amine;
[3-(4-Bromo-2-methyl-2H-pyrazol-3-yl)-4-methoxy-phenyl]-(4-chloro-phenyl)-amine;
[3-(4-Bromo-2-isopropyl-2H-pyrazol-3-yl)-phenyl]-(4-fluoro-phenyl)-amine;
[3-(4-Bromo-2-isopropyl-2H-pyrazol-3-yl)-phenyl]-(4-chloro-phenyl)-amine;
[3-(4-Bromo-2-methyl-2H-pyrazol-3-yl)-phenyl]-naphthalen-1-yl-amine; and
[3-(4-Bromo-2-methyl-2H-pyrazol-3-yl)-phenyl]-naphthalen-2-yl-amine;
Benzoxazol-2-yl-[3-(4-bromo-2-methyl-2H-pyrazol-3-yl)-phenyl]-amine;
[3-(4-Bromo-2-methyl-2H-pyrazol-3-yl)-phenyl]-quinolin-2-yl-amine;
(1H-Benzoinidazol-2-yl)-[3-(4-bromo-2-methyl-2H-pyrazol-3-yl)-phenyl]-amine;
Benzothiazol-2-yl-[3-(4-bromo-2-methyl-2H-pyrazol-3-yl)-phenyl]-amine;
[3-(4-Bromo-2-methyl-2H-pyrazol-3-yl)-phenyl]-(5-methoxy-benzothiazol-2-yl)-amine;
[3-(4-Bromo-2-methyl-2H-pyrazol-3-yl)-phenyl]-quinolin-3-yl -amine and
[3-(4-Bromo-2-methyl-2H-pyrazol-3-yl)-phenyl]-(5-chloro-benzothiazol-2-yl)-amine; or
a pharmaceutically acceptable salt, hydrate or solvate thereof.

117. A pharmaceutical composition comprising a compound according to claim 98 or 116, and a pharmaceutically acceptable carrier.

118. A method for prophylaxis or treatment of reducing platelet aggregation in an individual comprising administering to said individual in need of such prophylaxis or treatment a compound according to claim 98 or 116.

119. A method for prophylaxis or treatment of reducing a risk of blood clot formation in an angioplasty or coronary bypass surgery individual, comprising administering to said individual in need of such prophylaxis or treatment a compound according to claim 98 or 116.

120. A method for prophylaxis or treatment of reducing risk of blood clot formation in an individual suffering from atrial fibrillation, comprising administering to said individual in need of such prophylaxis or treatment a compound according to claim 98 or 116.

121. A method for prophylaxis or treatment of an individual suffering from at least one of the indications selected from the group consisting of behavioral disorder, drug induced psychosis, excitative psychosis, Gilles de la Tourette's syndrome, manic disorder, organic or NOS psychosis, psychotic disorder, psychosis, acute schizophrenia, chronic schizophrenia and NOS schizophrenia comprising administering to said individual in need of such prophylaxis or treatment a dopamine D2 receptor antagonist and a compound according to claim 98 or 116.

122. A method for prophylaxis or treatment of a sleep disorder in an individual comprising administering to said individual in need of such prophylaxis or treatment a compound according to claim 98 or 116.
